(19) United States
(12) Patent Application Publication
(10) Pub. No.: US 2006/0257418 A1
(43) Pub. Date: Nov. 16, 2006

(54) Vaccine

(75) Inventors: Lauren Bakaletz, Columbus, OH (US); Joseph Cohen, Rixensart (BE); Guy Dequesne, Rixensart (BE); Yves Lobet, Rixensart (BE)

Correspondence Address: GLAXOSMITHKLINE;Corporate Intellectual Property - UW2220, P.O. Box 1539, King of Prussia, PA, 19406-0939, US

(73) Assignees: SmithKline Beecham Biologicals, s.a.; Ohio State University Research Foundation

(21) Appl. No.: 11/337,733

(22) Filed: Jan. 23, 2006

Related U.S. Application Data
(63) Continuation of application No. 09/719,379, filed on Jun. 4, 2001, now abandoned.

(30) Foreign Application Priority Data
Jun. 11, 1998 (GB) 9812613.9

(51) Int. Cl. A61K 39/00; A61K 39/38

(52) U.S. Cl. 424/184.1

(57) ABSTRACT

This invention relates to three newly-identified, distinct groups of antigenic peptides [LB1(f) peptides] from the same region of the P5-like fimbrin protein discovered using sequence data from the fimbrin protein of many Haemophilus influenzae strains. The invention additionally provides chimeric polypeptides that carry one or more representatives of such peptides from different groups and which induce an immunogenic response in animals to Haemophilus influenzae. The peptides and polypeptides of the invention will be useful in vaccine compositions which provide protection against a wide range of H. influenzae strains.

This application is a continuation of application Ser. No. 09/719,379, filed 4 Jun. 2001, which is a 371 of International Application No. PCT/US99/11980, filed 28 May 1999, which claims priority of Great Britain Application No. 9812613.9, filed 11 Jun. 1998.

### FIELD OF INVENTION

This invention relates to newly identified peptides and polynucleotides encoding these peptides, and to chimeric proteins that carry these peptides. The invention also relates to a method of isolating the peptides or chimeric proteins and a vaccine composition for use in the treatment of Haemophilus influenzae infection.

### BACKGROUND OF THE INVENTION

Haemophilus influenzae (Hi) is a gram-negative coccobacillus and a strict human commensal. Strains of Hi are either encapsulated in a polysaccharide capsule or are non-encapsulated and are accordingly classified into typeable (encapsulated) and non-typeable (non-encapsulated) strains.

Encapsulated pathogenic strains of Hi cause mainly, but not exclusively, invasive disease in children under six years of age. Haemophilus influenzae type b (Hib), for example, is a major cause of meningitis and other invasive infections in children. Effective vaccines exist against Hib infections, and are based on producing antibodies to the polysaccharide capsule, and are therefore ineffective against non-typeable Haemophilus influenzae (ntHi).

Non-typeable Haemophilus influenzae (ntHi) represents the majority of the colonising strains and, although rarely invasive, are responsible for a significant proportion of mucosal disease including otitis media, sinusitis, chronic conjunctivitis and chronic or exacerbation of lower respiratory tract infections. Currently, approximately 30%, and as much as 62% of ntHi are resistant to penicillins. Carriage is estimated at 44% in children and approximately 5% in adults, and can persist for months. Neither the pathogenic mechanisms nor the host immunological response has been fully defined for otitis media caused by ntHi.

Otitis media is a common disease in children less than 2 years of age. It is defined by the presence of fluid in the middle ear accompanied by a sign of acute local or systemic illness. Acute signs include ear pain, ear drainage, hearing loss whereas systemic signs include fever, lethargy, irritability, anorexia, vomiting or diarrhoea. Streptococcus pneumoniae and non-typeable Haemophilus influenzae (ntHi) are the most predominant bacteria that cause the condition, accounting for 25-50%, and 15-30% of the species cultured, respectively. In addition, ntHi is responsible for 53% of recurrent otitis media. Approximately 60% and 80% of children have at least one episode of the disease by 1 and 3 years of age respectively (the peak being around 10 months).

There is evidence that protective immunity does exist for ntHi, however antigenic drift in the epitopes naturally involved (outer-membrane proteins P2, P4, P6) plays a major role in the ability of ntHi to evade the immune defence of the host.

There is therefore a need for additional effective vaccines against Haemophilus influenzae, and particularly for vaccines against non-typeable Haemophilus influenzae which is not affected by the currently available Hi polysaccharide vaccines.

Fimbriae, which are surface appendages found on ntHi, are produced in 100% of the bacteria recovered from the middle ears and nasopharyngeal region of children with chronic otitis media. A vaccine comprised of fimbrin, a filamentous protein derived from the fimbriae of ntHi has been reported (WO 94/26304). Fimbrin is homologous to the P5 outer membrane protein of ntHi that has been the subject of another patent application (EP 680765). The fimbrin P5-like protein is capable of eliciting antibodies that react to the bacteria's surface and are bactericidal (WO 94/26304). The protein has been purified and has been shown to induce an immune response against different strains of ntHi.

Existing methodologies to isolate fimbrin protein from the bacterial outer membrane are tedious and time-consuming. A strategy used with other bacterial species has been to produce relatively short linear peptides of the native protein. However, this approach has been of limited value since antibodies to such alternative immunogens frequently fail to recognise the native pathogen.

LB1(f) is a 19 amino-acid peptide (SEQ ID NO:5) derived from the sequence of P5-like fimbrin protein from strain ntHi1128 (occupying the region Arg117 to Gly135). This peptide was defined initially as being a potential B cell epitope, by analysis of the primary sequence of P5-like fimbrin protein. Immunising animals with chimeric fimbrin peptides (called LB1 peptides), comprising: the LB1(f) peptide; a linker peptide; and a T cell epitope, induces an immune response to the P5-like fimbrin protein and reduces the colonization of ntHi in animals subsequently exposed to ntHi (see U.S. Pat. No. 5,843,464). The LB1 peptide is immunogenic in vivo and antisera generated against it was immunoreactive against both denatured and native fimbriae. The peptide was thus able to act as an effective immunogen in that it was able to generate antibodies which recognised and bound to the epitope in its native structure. This is due in part to the synthetic LB1(f) peptide mimicking the coiled-coil secondary structure of the peptide within the fimbrin protein.

The problem with using protein antigens from only one strain of H. influenzae in a vaccine is that protection conferred tends to be largely restricted to homologous challenge [Bakaletz et al. (1997) Vaccine 15:955-961; Haase et al. (1991) Infect. Immun. 59:1278-1284; Sirakova et al. (1994) Infect. Immun. 62:2002-2020]. The antigenic diversity of the ntHi Outer Membrane Proteins, means that development of a broadly effective vaccine against a group of organisms as heterogeneous as ntHi will require a new strategy.

As will be seen, this invention relates to the more effective use of the LB1 (f) peptide as a vaccine against a broad spectrum of heterologous Haemophilus influenzae strains that express the P5-like fimbrin protein (or naturally occurring variants of the protein).

### SUMMARY OF THE INVENTION

It is an object of the present invention to provide groups of newly identified antigenic P5-like fimbrin subunit peptides (LB1(f) peptides) of P5-like fimbrin proteins from various ntHi strains. It is a further object to provide chimeric polypeptides that carry these peptides and which induce an immunogenic response in animals to ntHi, and polynucleotides encoding such peptides and polypeptides. The invention also relates to a method of isolating the peptides or chimeric polypeptides, to a method of detecting the presence of the peptides in biological samples, and to a vaccine composition for use in the treatment of Haemophilus influenzae infection.

The groups of LB1(f) peptides contain peptides from about 13 to about 22 amino acids in length. The peptides fall into 3 main groups (one of which contains 2 subgroups). The chimeric polypeptide comprises one or more of the LB1(f) peptide units covalently linked to a carrier protein that additionally acts as a T-cell epitope. Preferably the carrier protein is from Haemophilus influenzae so it may also induce an immunogenic response in animals to Haemophilus influenzae (including non-typeable Haemophilus influenzae).

The invention may be more fully understood by reference to the following drawings and detailed description.

### BRIEF DESCRIPTION OF THE DRAWINGS

FIG. 1: Plasmid pMGIMCS. The DNA sequence of the multiple cloning site is given in SEQ ID NO:73.

FIG. 2: Plasmid pRIT14588. The DNA sequence from the NdeI-XbaI site is given in SEQ ID NO:74 and the corresponding amino acid sequence is given in SEQ ID NO:75.

FIG. 3: Plasmid LPD-LB1-A. The DNA sequence from the BamHI-XbaI site is given in SEQ ID NO:76 and the corresponding amino acid sequence is given in SEQ ID NO:77.

FIG. 4: Plasmid LPD-LB1-II. The DNA sequence from the NdeI-XbaI site is given in SEQ ID NO:78 and the corresponding amino acid sequence is given in SEQ ID NO:79. The DNA and amino acid sequences of the Group 1 (LB1-GR1) and Group 2 (LB1-GR2) LB1(f) peptides are indicated with arrows. The arrows encompass the LB1 (f) within the sequence of its natural context within the p5-like fimbrin protein.

FIG. 5: Plasmid LPD-LB1-III. The DNA sequence from the BamHI-XbaI site is given in SEQ ID NO:80 and the corresponding amino acid sequence is given in SEQ ID NO:81. The DNA and amino acid sequences of the Group 1 (LB1-GR1), Group 2 (LB1-GR2), and Group 3 (LB1-GR3) LB1(f) peptides are indicated with arrows. The arrows encompass the LB1 (f) peptides within the sequence of its natural context within the p5-like fimbrin protein. The LB1(f) polypeptide (called LPD-LB1(f)_{2,1,3}) extends from Met1 to the C-terminal His residue before the stop codon.

FIG. 6: Acrylamide gel stained with Coomassie showing the expression products of the following plasmids:

Lanes: 1. MW markers 2. pMGMCS 3. pRIT14588

4. LPD-LB1-A 5. LPD-LB1-II6. LPD-LB1-III

7. LPD-LB1-III (LPD-LB1(f)_{2,1,3} after purification process) 8. MW markers

FIG. 7: Western Blot (using rabbit anti-LB1 antiserum) of an acrylamide gel showing the expression products of the following plasmids:

Lanes: 1. MW markers 2. pMGMCS 3. pRIT14588

4. LPD-LB1-A 5. LPD-LB1-II6. LPD-LB1-III

7. LPD-LB1-III (LPD-LB1(f)_{2,1,3} after purification process) 8. MW markers

FIG. 8: Western Blot (using a monoclonal anti-LPD antibody) of an acrylamide gel showing the expression products of the following plasmids:

Lanes: 1. MW markers 2. pMGMCS 3. pRIT14588

4. LPD-LB1-A 5. LPD-LB1-II6. LPD-LB1-III

7. LPD-LB1-III (LPD-LB1(f)_{2,1,3} after purification process) 8. MW markers

FIG. 9: Western Blot (using an antibody against the six-Histidine purification tag) of an acrylamide gel showing the expression products of the following plasmids:

Lanes: 1. MW markers 2. pMGMCS 3. pRIT14588

4. LPD-LB1-A 5. LPD-LB1-116. LPD-LB1-III

7. LPD-LB1-III (LPD-LB1(f)_{2,1,3} after purification process) 8. MW markers

FIG. 10: Passive transfer/challenge experiment. Mean tympanic membrane inflammation scores over the 35 day observation period for the 5 passively immunised chinchilla cohorts. The broken horizontal line at a mean tympanic membrane inflammation score of 1.5 indicates the level of inflammation attributable to adenovirus alone. Values above this line were considered to be an indication of ntHi-induced inflammation. ▾-Sham; ◯-LB1; ▪-LPD; ⋄-PD; Δ-LPD-LB1(f)_{2,1,3.}

FIG. 11: Bar graph showing the percentage of total middle ears known or suspected of containing an effusion based on otoscopy and tympanometry in five adenovirus-compromised chinchilla cohorts throughout the duration of the experiment. The time scale is measured with respect to the intranasal challenge of ntHi at day 0. Each animal received a 1:5 dilution of a specific antiserum by passive transfer prior to intranasal challenge with ntHi #86-028NP. Cohorts received antisera directed against:

[image]

FIG. 12: Western blot of serum used for passive transfer. Blot A=anti-LB1 serum pool. Blot B=anti-LPD-LB1(f)_{2,1,3} serum pool. Lanes contain: (1) molecular mass standards; (2) LPD; (3) LPD-LB1(f)_{2,1,3}; (4) LB1; (5) NTHi 86-028NP whole outer membrane protein (OMP) preparation; (6) NTHi 1885MEE whole OMP; (7) NTHi 1728MEE whole OMP.

FIG. 13: Study A. Passive transfer/challenge experiment. Mean tympanic membrane inflammation scores over the 35 day observation period for the 5 passively immunised chinchilla cohorts. Challenge was with either 86-028NP or 1885MEE strains of ntHi.

FIG. 14: Study B. Passive transfer/challenge experiment. Mean tympanic membrane inflammation scores over the 35 day observation period for the 5 passively immunised chinchilla cohorts. Challenge was with either 86-028NP or 1728MEE strains of ntHi.

FIG. 15: Study A. Chart showing the percentage of total middle ears known or suspected of containing an effusion based on otoscopy and tympanometry in six adenovirus-compromised chinchilla cohorts throughout the duration of the experiment. The time scale is measured with respect to the intranasal challenge of ntHi at day 0. Each animal received a 1:5 dilution of a specified antiserum by passive transfer prior to intranasal challenge with either ntHi #86-028NP or 1885MEE.

FIG. 16: Study B. Chart showing the percentage of total middle ears known or suspected of containing an effusion based on otoscopy and tympanometry in six adenovirus-compromised chinchilla cohorts throughout the duration of the experiment. The time scale is measured with respect to the intranasal challenge of ntHi at day 0. Each animal received a 1:5 dilution of a specified antiserum by passive transfer prior to intranasal challenge with either ntHi #86-028NP or 1728MEE.

### DETAILED DESCRIPTION OF THE PREFERRED EMBODIMENT

Peptides of the Invention

The peptides of the present invention relate to groups of newly identified LB1(f) peptides from P5-like fimbrin proteins of various ntHi strains from Europe and the United States.

The DNA sequence of the P5-like fimbrin protein was ascertained from 83 strains of ntHi, and the peptide sequence of the LB1(f) peptide was noted. The peptides of the present invention are B-cell epitopes which occurs in approximately the same region (and within the same context) of each protein—approximately in the region that encompasses positions 110 and 140 of the amino acid sequence of the protein. In strain ntHi-10567RM, for example, the peptide exists between Arg117 to Gly135 (SEQ ID NO:1).

After alignment, the peptide sequences of both the American and European ntHi strains fell into the same three groups, with some variation within these groups. Group 1 peptides [or LB1(f)₁] represented 71% of the peptides, contained about 19 amino acids, and had not less than 75% identity with the peptide provided in SEQ ID NO: 1. Group 2 peptides [or LB1 (f)₂] represented 19% of the peptides, contained 19-22 amino acids, and had not less than 75% identity with the peptide provided in SEQ ID NO:2. The group could be additionally divided into 2 subgroups, group 2a [or LB1(f)₂ₐ] exemplified by SEQ ID NO:2, and group 2b [or LB1(f)_{2b}] by SEQ ID NO:4. Group 3 peptides [or LB1(f)₃] represented 10% of the peptides, and contained 13 amino acids (provided in SEQ ID NO:3).

The sequence identity for peptides (and polypeptides and polynucleotides) can be calculated, for example, using the UWGCG Package which provides the BESTFIT program to calculate homology (identity), preferably on its default settings [Deveraux et al., Nucl. Acids Res. 12:387-395 (1984)].

Of 83 ntHi strains analysed, the LB1(f) peptides from all 62 US strains and all 21 European strains fell into Groups 1-3. Table 1 shows all ntHi strains that were analysed and which Group their respective LB1(f) peptides belong to. Tables 2, 3, and 4 list the cumulated sequences of Group 1, 2, and 3 LB1 (f) peptides respectively. Table 5 lists a representative example of a Group 1, 2a, 2b, and 3 LB1(f) peptide.

The previously known LB1(f) peptide (SEQ ID NO:5) falls into Group 1. Although it is known that this peptide is an effective immunogen, and confers protection against ntHi-caused otitis media, it has been unknown until now that this useful peptide exists in these three antigenically-distinct forms, which could be potentially combined to provide protective immunogens against all Haemophilus influenzae strains that express the P5-like fimbrin protein.

The peptides of this invention relate to the representative peptides of Groups 1, 2a, 2b, and 3 (SEQ ID NO: 1, 2, 4, and 3 respectively), and to antigenically related variants of these peptides. “Antigenically related variants” can be either natural variants (as exemplified by the peptides listed in tables 2, 3, and 4) or artificially modified variants that immunologically mimic the LB1(f) antigenic determinant site of the P5-like fimbrin protein. Such artificially modified variants can be made by synthetic chemistry or recombinant DNA mutagenesis techniques that are well known to persons skilled in the art (see for example Chapter 15 of Sambrook et al. “Molecular Cloning a Laboratory Manual” (1989) Cold Spring Harbor Laboratory Press). The antigenically related variants of the peptides should have an amino acid sequence identity of at least 75% to one of the peptides provided in SEQ ID NO: 1-4 (and more preferably at least 85%, and most preferably at least 95% identity), whilst still being capable of immunologically mimicking the corresponding antigenic determinant site of the P5-like fimbrin protein of non-typeable Haemophilus influenzae. For this invention “immunologically mimicking the corresponding antigenic determinant site of the P5-like fimbrin protein of ntHi” is defined as a (variant) peptide being capable of inducing antibodies that specifically recognises one of the wild-type LB1(f) sequences (listed in tables 2, 3, and 4) in the context of the whole P5-like fimbrin protein AND/OR defined as a (variant) peptide being capable of being recognised by the same immunospecific antibody that recognises one of the wild-type LB1(f) sequences (listed in tables 2, 3, and 4) in the context of the whole P5-like fimbrin protein. In the first definition, the variant peptide should be capable of inducing such antibodies either by itself, or in conjunction with a carrier molecule. In the second definition, the variant peptide should be capable of being recognised either by itself, or in conjunction with a carrier molecule. The antigenically related variant peptide does not include those peptides provided in SEQ ID NO: 5 (the previously determined LB1(f) peptide of P5-like fimbrin protein from strain ntHi-1128) and SEQ ID NO:6 (the previously determined LB1(f)-like peptide of P5 protein from ntHi).

Antigenically related variants may have had amino acids added, inserted, substituted or deleted. Preferred variants are those that differ from the referents by conservative (preferably single) amino acid substitutions.

The peptides of the invention also relates to combinations of LB1(f) peptides described above covalently linked, with optional spacer amino acids in between, to form a single peptide. For such combinations the peptides of SEQ ID NO: 5 & 6 can be used. The method to chemically synthesise or recombinantly express such peptides is well known to a person skilled in the art [see, for example, Sambrook et al. (1989)]. The optional spacer amino acids should preferably not be more than 18 amino acids either side of the peptide, and should preferably be composed of amino acids from the natural context of the LB1(f) peptide in the P5-like fimbrin protein (for example, if two LB1(f) peptides were joined, the first or N-terminal LB1(f) peptide could have 9 amino acids of its natural C-terminal context linked to 9 amino acids of the natural N-terminal context of the second or C-terminal LB1(f) peptide). One or more LB1(f) peptides may be linked in this way. Preferably 1-10 LB1(f) peptides are linked, more preferably 1-5, and still more preferably 1-3. More preferably, examples of at least one LB1(f) peptide from each LB1(f) group are linked in this way. Still more preferably, the LB1(f) peptides linked are those provided in SEQ ID NO: 2, 3, and 5. As the three antigenically-distinct peptides are combined, a more broadly protective immunogen is hence formed.

Polypeptides of the Invention

The polypeptides of the present invention relate to peptides described above being covalently linked to a carrier polypeptide that contains at least one T-cell epitope (for instance tetanus toxin, diptheria toxin, CRM197, Borrelia burgdorferi sensu lato OspA, Keyhole Limpet Haemocyanin, H. influenzae P6 protein, H. influenzae P5-like fimbrin protein, H. influenzae OMP26, H. influenzae protein D, or H. influenzae lipoprotein D) to form a chimeric LB1(f) polypeptide. This chimeric polypeptide comprises at least one of the LB1(f) peptides of the invention. Preferably the chimeric polypeptide comprises 1-10 LB1(f) peptides, more preferably 1-5, and still more preferably 1-3. These peptides can be linked N-terminally, C-terminally, or both N- and C-terminally to the carrier polypeptide. Preferably, the carrier polypeptide is from Haemophilus influenzae so that it can act as a good immunogenic carrier, whilst having some protective efficacy in itself and/or whilst providing a source of homologous T-cell epitopes derived from H. influenzae. Optionally, the chimeric polypeptide can also comprise a purification tag peptide sequence (such as a Histidine tag or a Glutathione-S-transferase tag) in order to aid subsequent purification of the polypeptide. Optional short peptide spacer sequences can be introduced between elements of the chimeric polypeptide (as defined above in the Peptides of the Invention).

Preferably, the carrier polypeptide used is OMP26 of H. influenzae (WO 97/01638), or protein P6 of H. influenzae (Nelson, M. B. et al., (1988) Infection and Immunity 56, 128-134).

Most preferably, the carrier polypeptide used is protein D (PD) from non-typeable Haemophilus influenzae or lipoprotein D (LPD—a lipidated form of PD). PD is a 42 kDa human IgD-binding outer surface protein that has been shown to be highly conserved among all strains of Haemophilus influenzae investigated so far (WO 91/18926). Both PD and LPD have been expressed in E. coli.

LPD was found to be a virulence factor in H. influenzae, and it elicits bactericidal activity against ntHi in rat antisera. LPD from H. influenzae and the recombinantly-expressed equivalent of LPD can thus act as a good immunogenic carrier, whilst having some protective efficacy in itself. The non-lipidated form (PD) is more conveniently used for process reasons, and is also a potential carrier polypeptide of this invention. LPD is very immunogenic because of its built-in adjuvant properties; that is, its ability to induce interleukins in macrophage and its ability to stimulate B cells to proliferate (WO 96/32963). PD does not have built-in adjuvant properties, and thus these conjugates are preferably adjuvanted, for example (but not limited) to aluminium hydroxide or aluminium phosphate. Antibody responses to LPD may protect against both typeable and nontypeable Hi strains. It thus represents an important carrier molecule for attaching other Hi antigens (such as LB1 (f) peptides) in order to obtain more effective vaccines against the organism. In addition to enhancing the immune response to the LB1(f) peptide antigen, LPD may serve as a protective antigen against both non-encapsulated and encapsulated strains of Hi.

Preferably three LB1(f) peptides are joined to the carrier polypeptide—one from each LB1(f) group. Preferably the LB1(f) peptides used are those provided in SEQ ID NO: 2, 3, and 5, and they are preferably linked C-terminally to the carrier polypeptide in the order SEQ ID NO: 2 (group 2 peptide), SEQ ID NO: 5 (group 1 peptide), SEQ ID NO: 3 (group 3 peptide). Such a polypeptide linked to LPD is known as LPD-LB1(f)_{2,1,3}. As the three antigenically-distinct peptides are combined, a more broadly protective immunogen is hence formed.

Although the chimeric polypeptide need not have a purification tag, when one is required a Histidine tag sequence is preferable, and it is preferably located at the C-terminus of the polypeptide.

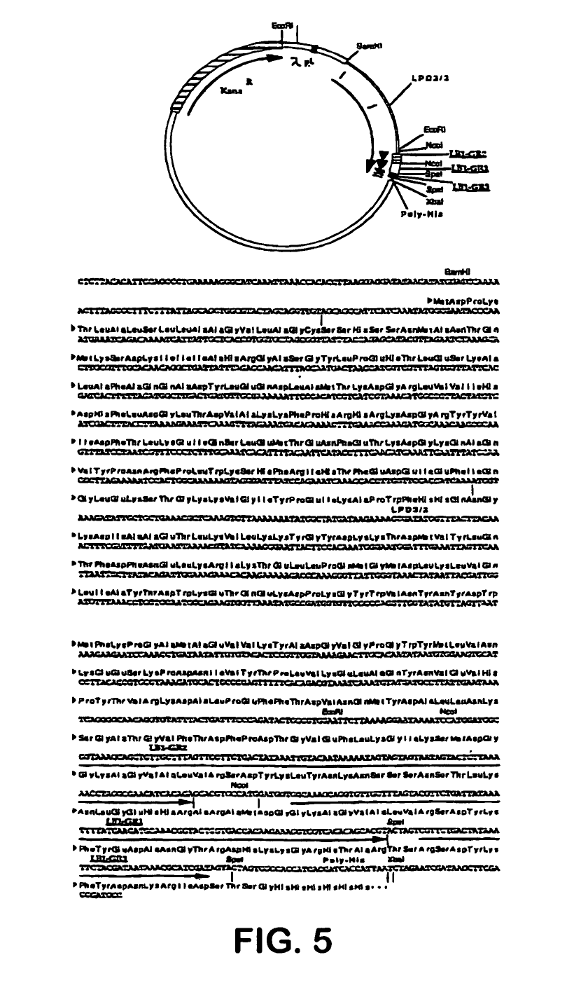

The sequence of a preferred LPD-LB1(f)_{2,1,3} chimeric polypeptide is provided in FIG. 5. Residues 1-19 is the signal sequence of Protein D. This signal peptide may be removed in order to produce the PD version of the chimeric polypeptide.

Polypeptides of the present invention can be prepared in any suitable manner. Such polypeptides include recombinantly produced polypeptides, synthetically produced polypeptides, or polypeptides produced by a combination of these methods. Means for preparing such polypeptides are well understood in the art, however examples of the method are presented in the Examples section.

Polynucleotides of the Invention

The polynucleotides of the present invention relates to the wild-type polynucleotide sequences of the LB1(f) peptides provided in Tables 6-8. They also relate to the wild-type DNA sequence of the polypeptides of the invention—that is to say constructing the chimeric polypeptide gene such that the wild-type gene sequence of the carrier polypeptide and wild-type polynucleotide sequences of LB1(f) peptides are used. Such a polynucleotide is provided in FIG. 5. The DNA sequence of the optional spacer amino acids is not essential for the invention, however where the spacer amino acids are from the natural context of the LB1(f) peptide, it is preferable (but not necessary) to use the natural DNA sequence of these spacers.

The polynucleotides of the invention also relates to DNA sequences that can be derived from the amino acid sequences of the peptides and polypeptides of the invention bearing in mind the degeneracy of codon usage. This is well known in the art, as is knowledge of codon usage in different expression hosts which is helpful in optimising the recombinant expression of the peptides and polypeptides of the invention.

The invention also provides polynucleotides which are complementary to all the above described polynucleotides.

When the polynucleotides of the invention are used for the recombinant production of polypeptides of the present invention, the polynucleotide may include the coding sequence for the mature polypeptide, by itself; or the coding sequence for the mature polypeptide in reading frame with other coding sequences, such as those encoding a leader or secretory sequence, a pre-, or pro- or prepro-protein sequence, or other fusion peptide portions (for instance amino acid residues 1 to 19 in FIG. 5, the natural signal sequence of LPD). For example, a marker sequence which facilitates purification of the fused polypeptide can be encoded. In certain preferred embodiments of this aspect of the invention, the marker sequence is a hexa-histidine peptide, as provided in the pQE vector (Qiagen, Inc.) and described in Gentz et al., Proc Natl Acad Sci USA (1989) 86:821-824, or is an HA tag, or is glutathione-s-transferase. Also preferred is LPD fused to its natural signal sequence (amino acid residues 1 to 19 in FIG. 5). The polynucleotide may also contain non-coding 5′ and 3′ sequences, such as transcribed, non-translated sequences, splicing and polyadenylation signals, ribosome binding sites and sequences that stabilize mRNA.

Vectors, Host Cells, Expression

The present invention also relates to vectors which comprise a polynucleotide or polynucleotides of the present invention, and host cells which are genetically engineered with vectors of the invention and to the production of peptides or polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Introduction of polynucleotides into host cells can be effected by methods described in many standard laboratory manuals, such as Davis et al., BASIC METHODS IN MOLECULAR BIOLOGY (1986) and Sambrook et al., MOLECULAR CLONING: A LABORATORY MANUAL, 2nd Ed., Cold Spring Harbor Laboratory Press, Cold Spring Harbor, N.Y. (1989) such as calcium phosphate transfection, DEAE-dextran mediated transfection, transvection, microinjection, cationic lipid-mediated transfection, electroporation, transduction, scrape loading, ballistic introduction or infection.

Representative examples of appropriate hosts include bacterial cells, such as meningococci, streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used. Such systems include, among others, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector suitable to maintain, propagate or express polynucleotides to produce a polypeptide in a host may be used. The appropriate nucleotide sequence may be inserted into an expression system by any of a variety of well-known and routine techniques, such as, for example, those set forth in Sambrook et al., MOLECULAR CLONING, A LABORATORY MANUAL (supra).

For secretion of the translated protein into the lumen of the endoplasmic reticulum, into the periplasmic space or into the extracellular environment, appropriate secretion signals may be incorporated into the desired polypeptide. These signals may be endogenous to the polypeptide (residues 1 to 19 in FIG. 5) or they may be heterologous signals.

Purification of Recombinantly Expressed Peptides/Polypeptides

Peptides and polypeptides of the invention can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during isolation and or purification.

Although the gene sequence of the chimeric LB1(f) polypeptide in the vector can be tagged with a Histidine-tag sequence which aids the purification of the polypeptide, it is not an essential element to the invention, as polypeptides without the Histidine-tag can still be purified by one of the techniques mentioned above.

Example 3 describes a purification method for purifying the LPD-LB1(f)(Group 2/Group 1/Group 3) (or LPD-LB1(f)_{2,1,3}) chimeric polypeptide. A LPD-LB1(f) chimeric polypeptide with three or more LB1(f) peptides at the C-terminus of the polypeptide is easier to purify over one with only a single LB1(f) peptide at the C-terminus. This is due to an observed partial degradation of the polypeptide from the C-terminus where it contains only one LB1(f) peptide that is not observed if there were three LB1(f) peptides at the C-terminus. Where some degradation has occurred, the full length polypeptide can be separated from the degraded form by incorporating a careful anion exchange step into the purification procedure.

Antibodies

The peptides and polypeptides of the invention, or cells expressing them can also be used as immunogens to produce antibodies immunospecific for the wild-type LB1(f) peptides. The term “immunospecific” means that the antibodies have substantially greater affinity for the peptides or polypeptides of the invention than their affinity for other related polypeptides in the prior art.

Antibodies generated against the peptides or polypeptides can be obtained by administering it to an animal, preferably a nonhuman, using routine protocols in the immunisation of an animal with an antigen, the collection of the blood, the isolation of the serum and the use of the antibodies that react with the peptide. The serum or IgG fraction containing the antibodies may be used in analysing the protein. For preparation of monoclonal antibodies, any technique which provides antibodies produced by continuous cell line cultures can be used. Examples include the hybridoma technique (Kohler, G. and Milstein, C., Nature (1975) 256:495-497), the trioma technique, the human B-cell hybridoma technique (Kozbor et al., Immunology Today (1983) 4:72) and the EBV-hybridoma technique (Cole et al., MONOCLONAL ANTIBODIES AND CANCER THERAPY, pp. 77-96, Alan R. Liss, Inc., 1985).

Techniques for the production of single chain antibodies (U.S. Pat. No. 4,946,778) can also be adapted to produce single chain antibodies to peptides or polypeptides of this invention. Also, transgenic mice, or other organisms including other mammals, may be used to express humanized antibodies.

The above-described antibodies may be employed to isolate or to identify clones expressing the peptide or to purify the peptides or polypeptides of the invention by affinity chromatography.

The peptides and polypeptides of the present invention also are useful to produce polyclonal antibodies for use in passive immunotherapy against H. influenzae. Human immunoglobulin is preferred because heterologous immunoglobulin may provoke a deleterious immune response to its foreign immunogenic components. Polyclonal antisera is obtained from individuals immunized with the peptides or polypeptides in any of the forms described. The immunoglobulin fraction is then enriched. For example, immunoglobulins specific for epitopes of the protein are enriched by immunoaffinity techniques employing the peptides or polypeptides of this invention. The antibody is specifically absorbed from antisera onto an immunoadsorbent containing epitopes of the polypeptide and then eluted from the immunoadsorbent as an enriched fraction of immunoglobulin.

Vaccines

The earlier work on the LB1(f) peptide from strain ntHi-1128 indicated that this peptide could be used as an immunogen for the development of a subunit vaccine against Haemophilus influenzae disease, particularly to prevent or reduce susceptibility to acute otitis media and other diseases caused by nontypeable strains. This invention extends this work by discovering three main Groups of LB1(f) peptides. The differences between the three groups are such that it is unlikely that efficient cross protection could be achieved between strains belonging to different groups. Therefore the present invention relies on the use of examples from each of these peptide groups to provide a more efficient and complete vaccine against strains of Haemophilus influenzae (preferably ntHi) that express the P5-like fimbrin protein.

Accordingly, another aspect of the invention is a vaccine composition comprising an immunogenic amount of at least one peptide or polypeptide of the invention. Preferably the composition should also comprise a pharmaceutically acceptable excipient. Vaccine preparation is generally described in Vaccine Design (“The subunit and adjuvant approach” (eds. Powell M. F. & Newman M. J). (1995) Plenum Press New York).

Additionally, the peptides and polypeptides of the present invention are preferably adjuvanted in the vaccine formulation of the invention. Suitable adjuvants include an aluminium salt such as aluminium hydroxide gel (alum) or aluminium phosphate, but may also be a salt of calcium, iron or zinc, or may be an insoluble suspension of acylated tyrosine, or acylated sugars, cationically or anionically derivatised polysaccharides, or polyphosphazenes. Other known adjuvants include CpG containing oligonucleotides. The oligonucleotides are characterised in that the CpG dinucleotide is unmethylated. Such oligonucleotides are well known and are described in, for example WO96/02555.

Further preferred adjuvants are those which induce an immune response preferentially of the TH1 type. High levels of Th1-type cytokines tend to favour the induction of cell mediated immune responses to the given antigen, whilst high levels of Th2-type cytokines tend to favour the induction of humoral immune responses to the antigen. Suitable adjuvant systems include, for example monophosphoryl lipid A, preferably 3-de-O-acylated monophosphoryl lipid A (3D-MPL), or a combination of 3D-MPL together with an aluminium salt. CpG oligonucleotides also preferentially induce a TH1 response. An enhanced system involves the combination of a monophosphoryl lipid A and a saponin derivative particularly the combination of QS21 and 3D-MPL as disclosed in WO 94/00153, or a less reactogenic composition where the QS21 is quenched with cholesterol as disclosed in WO 96/33739. A particularly potent adjuvant formulation involving QS21 3D-MPL & tocopherol in an oil in water emulsion is described in WO 95/17210 and is a preferred formulation.

Another aspect of the invention relates to a method for inducing an immunological response in a mammal which comprises inoculating the mammal with a peptide or polypeptide of the invention adequate to produce antibody and/or T cell immune response to protect said animal from H. influenzae disease, among others. Yet another aspect of the invention relates to a method of inducing immunological response in a mammal which comprises, delivering a peptide or polypeptide of the invention via a vector directing expression of a polynucleotide of the invention in vivo in order to induce such an immunological response to produce antibody to protect said animal from diseases.

A further aspect of the invention relates to an immunological/vaccine formulation (composition) which, when introduced into a mammalian host, induces an immunological response in that mammal to a LB1(f) peptide or polypeptide wherein the composition comprises a LB1(f) peptide or polypeptide gene, or LB1(f) peptide or polypeptide itself. The vaccine formulation may further comprise a suitable carrier. The LB1(f) vaccine composition is preferably administered orally, intranasally or parenterally (including subcutaneous, intramuscular, intravenous, intradermal, transdermal injection). Formulations suitable for parenteral administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant as described above. The dosage will depend on the specific activity of the vaccine and can be readily determined by routine experimentation.

Yet another aspect relates to an immunological/vaccine formulation which comprises the polynucleotide of the invention. Such techniques are known in the art, see for example Wolff et al., Science, (1990) 247: 1465-8.

The peptides or polypeptides of this invention can be administered as multivalent subunit vaccines in combination with antigens from other proteins of H. influenzae to achieve an enhanced bactericidal activity. They can also be administered in combination with polysaccharide antigens, for example the PRP capsular polysaccharide (preferably conjugated to a protein) of H. influenzae b. For combined administration with epitopes of other proteins, the LB1(f) peptide or polypeptide is either administered separately, as a mixture or as a conjugate or genetic fusion polypeptide. The conjugate is formed by standard techniques for coupling proteinaceous materials. The peptides or polypeptides of the invention can be used in conjunction with antigens of other organisms (e.g. encapsulated or nonencapsulated, bacteria, viruses, fungi and parasites). For example, the peptides or polypeptides of the invention are useful in conjunction with antigens of other microorganisms implicated in otitis media or other diseases. These include Streptococcus pneumoniae, Streptococcus pyrogenes group A, Staphylococcus aureus, respiratory syncytial virus and Branhemella catarrhalis.

As the polypeptides of the invention encompass the P5-like fimbrin protein itself, another preferred aspect of the invention is the combination of two or more P5-like fimbrin proteins from different LB1(f) groups in a vaccine formulation.

The evaluation of the peptides or polypeptides of the invention as potential vaccines against ntHi-caused otitis media is made in a chinchilla animal model developed by Dr. L. Bakaletz of Ohio State University. This model mimics the development of otitis media in children and is based on the successive intranasal administrations of adenovirus and ntHi a week apart. In these conditions, the bacteria is able, after the colonisation of the nasopharynx, to invade the middle ear via the Eustachian tube. Once there, ntHi will proliferate and induce an inflammatory process similar to what is observed in children.

For vaccine evaluation, by the time the chinchilla has been actively immunised they are too old at the time of challenge to be inoculated by the intranasal route with ntHi: even with a preinfection with adenovirus, almost none of them will develop otitis media. As an alternative route of challenge, a direct inoculation of the bacteria into the middle ear (bullae) through the skull is used. Passive transfer/challenge protocols can also be used to avoid needing trans-bullar challenge.

With all these types of challenge, the severity of the disease can be scored by otoscopic observation (through the external ear) or tympanometry, which evaluate the level of inflammation in the middle ear or changes in middle ear pressure and presence of fluid in the middle ear, respectively. The efficacy of a vaccine is determined by the reduction of the severity and/or the duration of the inflammation and the reduction of the colonisation in the ear and the nasopharynx.

In previous experiments, the protective efficacy of both LB1 from strain ntHi-1128 and LPD was evaluated after active immunisation, and intrabullar challenge. Repeatedly, immunisation with LB1 protected chinchilla against otitis media as indicated by a reduced length of otitis, reduced severity, and reduced colonisation in both the ears and the nasopharynx. The immunisation with LPD alone protected chinchillas against otitis media but not as well as LB1, and not reproducibly.

The vaccines of the invention can be further evaluated by examining whether the peptides or polypeptides of the invention inhibit adherence of ntHi to chinchilla epithelial throat cells, and whether they can prevent nasopharyngeal colonisation by nthi in vivo. The LB1 peptide from ntHi-1128 has a dose-dependent effect on the inhibition of the adherence of ntHi to chinchilla epithelial throat cells (probably as it acts as a direct steric inhibitor of ntHi binding), and lowers the ntHi in nasopharyngeal lavage fluid. Nasopharygeal colonisation is an initial step required for the development of otitis media, therefore this inhibition of colonisation will also help to inhibit the development of otitis media.

Diagnostic Assays/Kits

This invention also relates to the use of the peptides or polypeptides of the invention, and antibodies against these peptides or polypeptides as diagnostic reagents. Detection of a LB1(f) peptide will provide a diagnostic tool that can add to or define a diagnosis of Haemophilus influenzae disease, among others.

Biological samples for diagnosis may be obtained from a subject's cells, such as from serum, blood, urine, saliva, tissue biopsy, sputum, lavage fluids.

Polynucleotides of the invention, which are identical or sufficiently identical to one of the nucleotide sequences contained in Tables 6-8, may be used as hybridization probes for cDNA and genomic DNA or as primers for a nucleic acid amplification (PCR) reaction, to isolate full-length cDNAs and genomic clones encoding P5-like fimbrin protein. Such hybridization techniques are known to those of skill in the art. Typically these nucleotide sequences are 80% identical, preferably 90% identical, more preferably 95% identical to that of the referent. The probes generally will comprise at least 15 nucleotides. Preferably, such probes will have at least 30 nucleotides and may have at least 50 nucleotides. Particularly preferred probes will range between 30 and 50 nucleotides. In this way Haemophilus influenzae can be detected in a biological sample, and under particularly stringent hybridisation conditions, the specific strain or strains of Haemophilus influenzae present in a sample could be ascertained using the wild-type polynucleotide sequences provided in Tables 6-8.

Thus in another aspect, the present invention relates to a diagnostic kit for a disease, particularly Haemophilus influenzae disease, which comprises:

(a) a polynucleotide of the invention, preferably a nucleotide sequence provided in Tables 6-8;

(b) a nucleotide sequence complementary to that of (a);

(c) a LB1(f) peptide of the invention, preferably the peptides of SEQ ID NO: 1-4; or

(d) an antibody to a LB1(f) peptide of the invention, preferably to the peptides of SEQ ID NO: 1-4.

It will be appreciated that in any such kit, (a), (b), (c) or (d) may comprise a substantial component.

Cited documents are incorporated by reference herein.

The invention is further illustrated by the following examples.

### EXAMPLES

The examples below are carried out using standard techniques, which are well known and routine to those skilled in the art, except where otherwise described in detail. The examples illustrate, but do not limit the invention.

### Example 1

### The determination of the amino acid sequence variability of the LB1(f) peptide in various ntHi strains.

1 a) Culture of ntHi Isolates—the Preparation of Samples for PCR Analysis

53 ntHi isolates were obtained from Dr. L. Bakaletz of Ohio State University, and 30 ntHi isolates were obtained from Dr. A. Forsgren of Mälmo, Sweden.

0.1 mL of a liquid culture of each ntHi isolate was spread on Gelose Chocolate Agar (GCA). The purity of the samples was controlled on solidified media (TSA—Tryptose Soy Agar in Petri dishes). The dishes were incubated at 35° C. for 24 hours. Colonies from dishes were resuspended in 5 mL of filtered TSB (Tryptose Soy Broth+3 μg/μl NAD;+3 μg/μl Hémine, +1% horse serum). 50 mL of TSB liquid media was inoculated with 2.5 mL of the culture, and were incubated at 35° C. When the concentration of the culture grew to 10⁸ cells/mL, 10 mL of culture were centrifuged at 10,000 rpm, 4° C. for 15 minutes. The supernatant was removed and the cells were washed in physiological buffer. The cells were centrifuged at 10,000 rpm for 15 minutes, 4° C. The cells were resuspended at a final concentration of 10⁹ cells/mL. The cells were boiled at 95-100° C. for 10-15 minutes, and then placed directly on ice. Samples were frozen at −70° C. The samples were then ready for DNA amplification by PCR.

1b) Amplification of P5-Like Fimbrin Gene DNA Fragment by PCR

PCR amplification of fragment of the fimbrin gene were performed on the ntHi preparations from example 1a). 200 μL of an ntHi preparation were centrifuged 14,200 rpm for 3 minutes at room temperature. All the supernatant was removed. The cells were resuspended in 25 μL of ADI, were boiled at 95° C. for 10 minutes, and were centrifuge for 3 minutes at 14,200 rpm. 5 μL of supernatant were used for a PCR reaction.

Amplification of DNA was performed with specific primers:

| | |
|---|---|
| NTHi-01: | |
| 5′-ACT-GCA-ATC-GCA-TTA-GTA-GTT-GC-3′ | SEQ ID NO:56 |
| NTHi-02: | |
| 5′-CCA-AAT-GCG-AAA-GTT-ACA-TCA-G-3′ | SEQ ID NO:57 |

The PCR reaction mixture was composed of the following: cell extract supernatant, 5.0 μL; Primer NTHi-01 ( 1/10), 1.0 μL; Primer NTHi-02 ( 1/10), 1.0 μL; DMSO, 2.0 μL; dNTP mix, 4.0 μL; Buffer 10×, 5.0 μL; ADI, 31.5 μL; Taq polymerase, 0.5 μL.

The PCR cycle conditions were as follows: (94° C. for 1 min; 50° C. for 1 min; 72° C. for 3 min) for 25 cycles, and finishing with 72° C. for 10 min. The reaction was monitored by electrophoresis in a 3% agarose gel in TBE buffer.

The primers used for the identification of which group a particular ntHi P5-like fimbrin LB1(f) peptide belonged to are as follows (they are used in a similar way to the reaction above):

| | |
|---|---|
| Group 1: | |
| NTHi-01: | |
| 5′-ACT-GCA-ATC-GCA-TTA-GTA-GTT-GC-3′ | SEQ ID NO:56 |
| NTHi-GR1: | |
| 5′-GTG-GTC-ACG-AGT-ACC-G-3′ | SEQ ID NO:58 |
| Group 2: | |
| NTHi-01: | |
| 5′-ACT-GCA-ATC-GCA-TTA-GTA-GTT-GC-3′ | SEQ ID NO:56 |
| NTHi-GR2bis: | |
| 5′-TCT-GTG-ATG-TTC-GCC-TAG-3′ | SEQ ID NO:59 |
| Group 3: | |
| NTHi-01: | |
| 5′-ACT-GCA-ATC-GCA-TTA-GTA-GTT-GC-3′ | SEQ ID NO:56 |
| NTHi-GR3: | |
| 5′-CTA-TCG-ATG-CGT-TTA-TTA-TC-3′ | SEQ ID NO:60 |

1c) DNA Purification

The PCR Clean Up Kit for purification of DNA fragments from PCR reactions was used (Boehringer Mannheim). At the end of the procedure, the purified PCR product was eluted twice in 25 μL volumes of redistilled water from the silica resin.

The purified products were analyzed by electrophoresis in a 3% agarose gel stained with ethidium bromide. The DNA was then ready for sequencing.

1 d) DNA Sequencing

This was done using an ABI Automatic Sequence, the ABI-PRISM—DNA sequencing Kit (using Terminator PCR Cycle Sequencing), and Amplitaq DNA Polymerase FS (from Perkin Elmer).

The PCR reaction mixture used was as follows: Mix (from the kit), 8.0 μL; DNA (approx. 1 μg), 3.0 μL; Primer (see below) ⅕ or 1/10, 1.0 μL; ADI, 8.0 μL

The sequencing primers used were as follows:

| | |
|---|---|
| NTHi-03: | |
| 5′-AGG-TTA-CGA-CGA-TTT-CGG-3′ | SEQ ID NO:61 |
| or | |
| NTHi-04: | |
| 5′-CGC-GAG-TTA-GCC-ATT-GG-3′ | SEQ ID NO:62 |
| or | |
| NTHi-05: | |
| 5′-AAA-GCA-GGT-GTT-GCT-TTA-G-3′ | SEQ ID NO:63 |
| or | |
| NTHi-06: | |
| 5′-TAC-TGC-GTA-TTC-TTA-TGC-ACC-3′ | SEQ ID NO:64 |
| OR | |
| NTHi-03: | |
| 5′-AGG-TTA-CGA-CGA-TTT-CGG-3′ | SEQ ID NO:61 |
| NTHi-04: | |
| 5′-CGC-GAG-TTA-GCC-ATT-GG-3′ | SEQ ID NO:62 |
| NTHi-05: | |
| 5′-AAA-GCA-GGT-GTT-GCT-TTA-G-3′ | SEQ ID NO:63 |
| NTHi-06: | |
| 5′-TAC-TGC-GTA-TTC-TTA-TGC-ACC-3′ | SEQ ID NO:64 |
| NTHi-14: | |
| 5′-GGT-GTA-TTT-GGT-GGT-TAC-C-3′ | SEQ ID NO:65 |
| NTHi-15: | |
| 5′-GTT-ACG-ACG-ATT-ACG-GTC-G-3′ | SEQ ID NO:66 |

The PCR cycle sequencing conditions were as follows: (96° C. for 30 seconds; 50° C. for 15 seconds; 60° C. for 4 min) for 25 cycles, and finishing with 72° C. for 10 mm.

The PCR product was prepared and analysed by: adding 80 μL ADI to the PCR sequence reaction to obtain a final volume of 100 μL; adding an equal volume of phenol/chloroform to the DNA solution. The sample was then centrifuged at 14,500 rpm at 4° C. for 3 min and the top aqueous layer was removed. The phenol/chloroform step and the centrifugation step were repeated once more. 10 μL 3M NaAc pH 4.8 and 220 μL 100% ethanol (at room temperature) were then added and mixed. The sample was placed at −20° C. for 5 min, and then centrifuged at 14,000 rpm 20 min at 4° C. The ethanol supernatant was removed and the pellet was rinsed with 1 mL of 70% ethanol (at room temperature). This was centrifuged at 14,000 rpm 10 min at 4° C., and the supernatant was removed as before. The pellet was air dried, and frozen overnight. The pellet was dissolved in the following solution: formamide 100% deionised water, 5 volumes; 0.5M EDTA pH 8.00, 1 volume. This was vortexed a few seconds and loaded on a sequencing gel.

1 e) Cumulated Results and Conclusions

A list of the various ntHi isolates that were analysed in terms of the sequence of their LB1(f) peptides from P5-like fimbrin protein is shown in Table 1. The group classification was determined by aligning the LB1(f) peptide against SEQ ID NO: 5, 2, or 3 (being the representative Group 1, 2 or 3 LB1(f) peptides respectively). LB1 (f) peptides had to have at least 75% identity with the representative peptide of a group in order for the classification of the group to be assigned to the test peptide. Tables 2, 3, and 4 show the aligned sequences of the Group 1, 2, and 3 LB1(f) peptide sequences respectively. Table 5 shows the representative LB1(f) peptides of Group 1, 2a, 2b, and 3 aligned with respect to each other.

Tables 6-9 show the DNA sequences of the LB1(f) peptides of Tables 2-5, respectively.

TABLE 1

| | Serotype | n^{o} order | Strains | Group | | |
|---|---|---|---|---|---|---|
| 1 | NTHi | 1848L | H. influenzae | 1 | | |
| 2 | NTHi | 1848NP | H. influenzae | 1 | | |
| 3 | NTHi | 1885R | H. influenzae | 1 | | |
| 4 | NTHi | 1885MEE | H. influenzae | | 2 | |
| 5 | NTHi | 10547RMEE | H. influenzae | | | 3 |
| 6 | NTHi | 10548LMEE | H. influenzae | | | 3 |
| 7 | NTHi | 10567RMEE | H. influenzae | 1 | | |
| 8 | NTHi | 10568LMEE | H. influenzae | 1 | | |
| 9 | NTHi | 10567&8NP | H. influenzae | | | 3 |
| 10 | NTHi | 1371MEE | H. influenzae | 1 | | |
| 11 | NTHi | 214NP | H. influenzae | 1 | | |
| 12 | NTHi | 1370MEE | H. influenzae | 1 | | |
| 13 | NTHi | 1380MEE | H. influenzae | 1 | | |
| 14 | NTHi | 217NP | H. influenzae | 1 | | |
| 15 | NTHi | 266NP | H. influenzae | | 2 | |
| 16 | NTHi | 167NP | H. influenzae | 1 | | |
| 17 | NTHi | 1657MEE | H. influenzae | 1 | | |
| 18 | NTHi | 284NP | H. influenzae | 1 | | |
| 19 | NTHi | 1666MEE | H. influenzae | 1 | | |
| 20 | NTHi | 287NP | H. influenzae | 1 | | |
| 21 | NTHi | 1236MEE | H. influenzae | | 2 | |
| 22 | NTHi | 183NP | H. influenzae | | 2 | |
| 23 | NTHi | 165NP | H. influenzae | | 2 | |
| 24 | NTHi | 1182MEE | H. influenzae | 1 | | |
| 25 | NTHi | 166NP | H. influenzae | 1 | | |
| 26 | NTHi | 1199MEE | H. influenzae | 1 | | |
| 27 | NTHi | 172NP | H. influenzae | 1 | | |
| 28 | NTHi | 1230MEE | H. influenzae | 1 | | |
| 29 | NTHi | 180NP | H. influenzae | 1 | | |
| 30 | NTHi | 1234MEE | H. influenzae | 1 | | |
| 31 | NTHi | 182NP | H. influenzae | 1 | | |
| 32 | NTHi | 152NP | H. influenzae | 1 | | |
| 33 | NTHi | 226NP | H. influenzae | 1 | | |
| 34 | NTHi | 1714MEE | H. influenzae | | 2 | |
| 35 | NTHi | 297NP | H. influenzae | | 2 | |
| 36 | NTHi | 1715MEE | H. influenzae | | 2 | |
| 37 | NTHi | 1729MEE | H. influenzae | | | 3 |
| 38 | NTHi | 1728MEE | H. influenzae | | | 3 |
| 39 | NTHi | 250NP | H. influenzae | 1 | | |
| 40 | NTHi | 1563MEE | H. influenzae | 1 | | |
| 41 | NTHi | 1562MEE | H. influenzae | 1 | | |
| 42 | NTHi | 10559RMEE | H. influenzae | 1 | | |
| 43 | NTHi | 1712MEE | H. influenzae | 1 | | |
| 44 | NTHi | 1521 | H. influenzae | 1 | | |
| 45 | NTHi | 1060RMEE | H. influenzae | 1 | | |
| 46 | NTHi | 86-027MEE | H. influenzae | | 2 | |
| 47 | NTHi | 86-027NP | H. influenzae | 1 | | |
| 48 | NTHi | 86-028NP | H. influenzae | 1 | | |
| 49 | NTHi | 86-028LMEE | H. influenzae | 1 | | |
| 50 | NTHi | 90-100 | H. influenzae | 1 | | |
| 51 | NTHi | 90-121RMEE | H. influenzae | 1 | | |
| 52 | NTHi | 1128 | H. influenzae | 1 | | |
| 53 | NTHi | 90-100RMEE | H. influenzae | 1 | | |
| 54 | NTHi* | 476 | H. influenzae | 1 | | |
| 55 | NTHi* | 480 | H. influenzae | 1 | | |
| 56 | NTHi* | 481 | H. influenzae | 1 | | |
| 57 | NTHi* | 482 | H. influenzae | 1 | | |
| 58 | NTHi* | 484 | H. influenzae | 1 | | |
| 59 | NTHi* | 486 | H. influenzae | 1 | | |
| 60 | NTHi* | 490 | H. influenzae | 1 | | |
| 61 | NTHi* | 492 | H. influenzae | | 2 | |
| 62 | NTHi* | 494 | H. influenzae | 1 | | |
| 63 | NTHi* | 495 | H. influenzae | | 2 | |
| 64 | NTHi* | 498 | H. influenzae | 1 | | |
| 65 | NTHi* | 499 | H. influenzae | 1 | | |
| 66 | NTHi* | 500 | H. influenzae | | 2 | |
| 67 | NTHi* | 501 | H. influenzae | 1 | | |
| 68 | NTHi* | 502 | H. influenzae | | 2 | |
| 69 | NTHi* | 503 | H. influenzae | 1 | | |
| 70 | NTHi* | 504 | H. influenzae | | | 3 |
| 71 | NTHi* | 506 | H. influenzae | | 2 | |
| 72 | NTHi* | 507 | H. influenzae | 1 | | |
| 73 | NTHi* | 546 | H. influenzae | | 2 | |
| 74 | NTHi* | 567 | H. influenzae | 1 | | |
| 75 | NTHi | 544 | H. influenzae | | | 3 |
| 76 | NTHi | 565 | H. influenzae | 1 | | |
| 77 | NTHi | 600 | H. influenzae | | | 3 |
| 78 | NTHi | 601 | H. influenzae | 1 | | |
| 79 | NTHi | 603 | H. influenzae | 1 | | |
| 80 | NTHi | 604 | H. influenzae | | 2 | |
| 81 | NTHi | 605 | H. influenzae | 1 | | |
| 82 | NTHi | 606 | H. influenzae | 1 | | |
| 83 | NTHi | 608 | H. influenzae | 1 | | |

Cumulated list of ntHi strains investigated and the classification of the sequence of their respective LB1(f) peptides from P5-like fimbrin protein (strains 1-53 from L. Bakaletz, strains 54-83 from A. Forsgren). * denotes a European strain of ntHi, all others were isolated from the United States. Strains 1885 and 1128 are available from the American Type Culture Collection (ATCC # 55431 and 55430 respectively).

TABLE 2
Cumulated Group 1 Peptide Sequences

| | | |
|---|---|---|
| N1128 | RSDYKFYEDANGTRDHKKG | SEQ ID NO:5 |
| N1380MEE | RSDYKFYEDANGTRDHKKG | SEQ ID NO:5 |
| N1885R | RSDYKFYEDANGTRDHKKG | SEQ ID NO:5 |
| N1562MEE | RSDYKFYEDANGTRDHKKG | SEQ ID NO:5 |
| N1563MEE | RSDYKFYEDANGTRDHKKG | SEQ ID NO:5 |
| N180NP | RSDYKFYEDANGTRDHKKG | SEQ ID NO:5 |
| N217NP | RSDYKFYEDANGTRDHKKG | SEQ ID NO:5 |
| N284NP | RSDYKFYEDANGTRDHKKG | SEQ ID NO:5 |
| N1666MEE | RSDYKFYEDANGTRDHKKG | SEQ ID NO:5 |
| N1230MEE | RSDYKFYEDANGTRDHKKG | SEQ ID NO:5 |
| NTHI-501 | RSDYKFYEDANGTRDHKKG | SEQ ID NO:5 |
| NTHI-507 | RSDYKFYEDANGTRDHKKG | SEQ ID NO:5 |
| NTHI-565 | RSDYKFYEDANGTRDHKKG | SEQ ID NO:5 |
| NTHI-603 | RSDYKFYEDANGTRDHKKG | SEQ ID NO:5 |
| NTHI-608 | RSDYKFYEDANGTRDHKKG | SEQ ID NO:5 |
| N287NP | RSDYKFYEDANGTRDHKKG | SEQ ID NO:5 |
| N86028LM | RSDYKFYEDANGTRDHKKG | SEQ ID NO:5 |
| N86028NP | RSDYKFYEDANGTRDHKKG | SEQ ID NO:5 |
| N152NP | RSDYKFYEDADGTRDHKKG | SEQ ID NO:7 |
| N1234MEE | RSDYKFYDDANGTRDHKKG | SEQ ID NO:8 |
| N182NP | RSDYKFYDDANGTRDHKKG | SEQ ID NO:8 |
| N90100RM | RSDYKFYEDENGTRDHKKG | SEQ ID NO:9 |
| N90100 | RSDYKFYEDENGTRDHKKG | SEQ ID NO:9 |
| N10567RM | RSDYKFYEAANGTRDHKKG | SEQ ID NO:1 |
| N1060MEE | RSDYKFYEAANGTRDHKKG | SEQ ID NO:1 |
| N172NP | RSDYKFYEAANGTRDHKKG | SEQ ID NO:1 |
| N1199MEE | RSDYKFYEAANGTRDHKKG | SEQ ID NO:1 |
| N10568LM | RSDYKFYEAANGTRDHKKG | SEQ ID NO:1 |
| N90121RM | RSDYKFYEAANGTRDHKKG | SEQ ID NO:1 |
| N86027NP | RSDYKFYEVANGTRDHKKG | SEQ ID NO:10 |
| NTHI-486 | RSDYKFYEVANGTRDHKKG | SEQ ID NO:10 |
| N1712MEE | RSDYKFYEVANGTRDHKKG | SEQ ID NO:10 |
| NTHI-503 | RSDYKFYEAANGTRDHKKG | SEQ ID NO:1 |
| NTHI-476 | RSDYKFYEEANCTRDHKKG | SEQ ID NO:11 |
| N166NP | RSDYKFYNDANGTRDHKKS | SEQ ID NO:12 |
| N1182MEE | RSDYKFYNDANGTRDHKKS | SEQ ID NO:12 |
| N1848NP | RSDYKFYEVANGTRDHKKS | SEQ ID NO:13 |
| N1371MEE | RSDYKFYEVANGTRDHKKS | SEQ ID NO:13 |
| NTHI-498 | RSDYKFYEVANGTRDHKKS | SEQ ID NO:13 |
| NTHI-606 | RSDYKFYEVANGTRDHKKS | SEQ ID NO:13 |
| N1848L | RSDYKFYEVANGTRDHKKS | SEQ ID NO:13 |
| NTHI-567 | RSDYKFYEDANGTRDRKTG | SEQ ID NO:14 |
| NTHI-484 | RSDYKFYEDANGTRKHKEG | SEQ ID NO:15 |
| N10559RM | RSDYKLYEVANGTRDHKKS | SEQ ID NO:16 |
| NTHI-601 | RSDYKFYEVANGTRDHKQS | SEQ ID NO:17 |
| NTHI-481 | RSDYKFYEVANGTRDHKQS | SEQ ID NO:17 |
| NTHI-482 | RSDYKFYEVANGTRDHKQS | SEQ ID NO:17 |
| N1370MEE | RSDYKFYEVANGTRDHKQS | SEQ ID NO:17 |
| N226NP | RSDYKFYEEANGTRDHKRS | SEQ ID NO:18 |
| NTHI-480 | RSDYKFYEDANGTRERKRG | SEQ ID NO:19 |
| N1657MEE | RSDYKFYEVANGTRERKKG | SEQ ID NO:20 |
| N267NP | RSDYKFYEVANGTRERKKG | SEQ ID NO:20 |
| NTHI-490 | RSDYKFYEVANGTRERKKG | SEQ ID NO:20 |
| NTHI-494 | RSDYKFYEVANGTRERKKG | SEQ ID NO:20 |
| N214NP | RSDYKFYEVPNGTRDHKQS | SEQ ID NO:21 |
| N250NP | RSDYKRYEEANGTRNHDKG | SEQ ID NO:22 |
| N1521 | RSDYKRYEEANGTRNHDKG | SEQ ID NO:22 |
| NTHI-605 | RSDYKRYEEANGTRNHDKG | SEQ ID NO:22 |
| NTHI-499 | RSDYEFYEAPNSTRDHKKG | SEQ ID NO:23 |

TABLE 3
Cumulated Group 2 Peptide Sequences

| | | |
|---|---|---|
| N1715MEE | RSDYKLYNKNSSSNSTLKNLGE | SEQ ID NO:2 |
| N1714MEE | RSDYKLYNKNSSSNSTLKNLGE | SEQ ID NO:2 |
| N86027RM | RSDYKLYNKNSSSNSTLKNLGE | SEQ ID NO:2 |
| N297NP | RSDYKLYNKNSSSNSTLKNLGE | SEQ ID NO:2 |
| N266NP | RSDYKLYNKNSSSNSTLKNLGE | SEQ ID NO:2 |
| N1885MEE | RSDYKLYNKNSSSNSTLKNLGE | SEQ ID NO:2 |
| NTHI-546 | RSDYKLYNKNSSSNSTLKNLGE | SEQ ID NO:2 |
| NTHI-604 | RSDYKLYNKNSSSNSTLKNLGE | SEQ ID NO:2 |
| NTHI-492 | RSDYKLYNKNSS-NSTLKNLGE | SEQ ID NO:24 |
| NTHI-502 | RSDYKLYDKNSSSN-TLKKLGE | SEQ ID NO:25 |
| NTHI-506 | RSDYKLYNKNSS-NSTLKNLGE | SEQ ID NO:26 |
| N1236MEE | RSDYKLYNKNSS---TLKDLGE | SEQ ID NO:4 |
| NTHI-500 | RSDYKLYNKNSS---TLKDLGE | SEQ ID NO:4 |
| NTHI-183 | RSDYKLYNXNSS---TLKDLGE | SEQ ID NO:4 |
| N165NP | RSDYKLYNKNSSN-TLKDLGE | SEQ ID NO:26 |
| NTHI-495 | RSDYKLYNKNSSD-ALKKLGE | SEQ ID NO:27 |

TABLE 4
Cumulated Group 3 Peptide Sequences

| | | |
|---|---|---|
| N1729MEE | RSDYKFYDNKRID | SEQ ID NO:3 |
| NTHI-504 | RSDYKFYDNKRID | SEQ ID NO:3 |
| NTHI-544 | RSDYKFYDNKRID | SEQ ID NO:3 |
| NTHI-600 | RSDYKFYDNKRID | SEQ ID NO:3 |
| N1728MEE | RSDYKFYDNKRID | SEQ ID NO:3 |
| N10548LM | RSDYKFYDNKRID | SEQ ID NO:3 |
| N10547RM | RSDYKFYDNKRID | SEQ ID NO:3 |
| N105678R | RSDYKFYDNKRID | SEQ ID NO:3 |

TABLE 5

Cumulated Group 1, 2a, 2b, and 3 Peptide
Sequences

| | | |
|---|---|---|
| N1128 | RSDYKFYEDANGTRDHKKG--- | SEQ ID NO:5 |
| N1715MEE | RSDYKLYNKNSSSNSTLKNLGE | SEQ ID NO:2 |
| NTHI-183 | RSDYKLYNKNSS---TLKDLGE | SEQ ID NO:4 |
| N1729MEE | RSDYKFYDN------KRID--- | SEQ ID NO:3 |

TABLE 6
Cumulated Group 1 Gene Sequences

| | | |
|---|---|---|
| N1128 | CGTTCTGATTATAAATTTTATGAAGATGC | SEQ ID NO:29 |
| | AAACGGTACTCGTGACCACAAGAAAGGT | |
| N1380MEE | CGTTCTGATTATAAATTTTATGAAGATGC | SEQ ID NO:29 |
| | AAACGGTACTCGTGACCACAAGAAAGGT | |
| N1885R | CGTTCTGATTATAAATTTTATGAAGATGC | SEQ ID NO:29 |
| | AAACGGTACTCGTGACCACAAGAAAGGT | |
| N1562MEE | CGTTCTGATTATAAATTTTATGAAGATGC | SEQ ID NO:29 |
| | AAACGGTACTCGTGACCACAAGAAAGGT | |
| N1563MEE | CGTTCTGATTATAAATTTTATGAAGATGC | SEQ ID NO:29 |
| | AAACGGTACTCGTGACCACAAGAAAGGT | |
| N180NP | CGTTCTGATTATAAATTTTATGAAGATGC | SEQ ID NO:29 |
| | AAACGGTACTCGTGACCACAAGAAAGGT | |
| N217NP | CGTTCTGATTATAAATTTTATGAAGATGC | SEQ ID NO:29 |
| | AAACGGTACTCGTGACCACAAGAAAGGT | |
| N284NP | CGTTCTGATTATAAATTTTATGAAGATGC | SEQ ID NO:29 |
| | AAACGGTACTCGTGACCACAAGAAAGGT | |
| N1666MEE | CGTTCTGATTATAAATTTTATGAAGATGC | SEQ ID NO:29 |
| | AAACGGTACTCGTGACCACAAGAAAGGT | |
| N1230MEE | CGTTCTGATTATAAATTTTATGAAGATGC | SEQ ID NO:29 |
| | AAACGGTACTCGTGACCACAAGAAAGGT | |
| NTHI-501 | CGTTCTGATTATAAATTTTATGAAGATGC | SEQ ID NO:29 |
| | AAACGGTACTCGTGACCACAAGAAAGGT | |
| NTHI-507 | CGTTCTGATTATAAATTTTATGAAGATGC | SEQ ID NO:29 |
| | AAACGGTACTCGTGACCACAAGAAAGGT | |
| NTHI-565 | CGTTCTGATTATAAATTTTATGAAGATGC | SEQ ID NO:29 |
| | AAACGGTACTCGTGACCACAAGAAAGGT | |
| NTHI-603 | CGTTCTGATTATAAATTTTATGAAGATGC | SEQ ID NO:29 |
| | AAACGGTACTCGTGACCACAAGAAAGGT | |
| NTHI-608 | CGTTCTGATTATAAATTTTATGAAGATGC | SEQ ID NO:29 |
| | AAACGGTACTCGTGACCACAAGAAAGGT | |
| N287NP | CGTTCTGATTATAAATTTTATGAAGATGC | SEQ ID NO:29 |
| | AAACGGTACTCGTGACCACAAGAAAGGT | |
| N86028LM | CGTTCTGATTATAAATTTTATGAAGATGC | SEQ ID NO:29 |
| | AAACGGTACTCGTGACCACAAGAAAGGT | |
| N86028NP | CGTTCTGATTATAAATTTTATGAAGATGC | SEQ ID NO:29 |
| | AAACGGTACTCGTGACCACAAGAAAGGT | |
| N152NP | CGTTCTGATTATAAATTTTATGAAGATGC | SEQ ID NO:30 |
| | AGACGGTACTCGTGACCACAAGAAAGGT | |
| N1234MEE | CGTTCTGATTATAAATTTTATGATGATGC | SEQ ID NO:31 |
| | AAACGGTACTCGTGACCACAAGAAAGGT | |
| 182NP | CGTTCTGATTATAAATTTTATGATGATGC | SEQ ID NO:31 |
| | AAACGGTACTCGTGACCACAAGAAAGGT | |
| N90100RM | CGTTCTGATTATAAATTTTATGAAGATGA | SEQ ID NO:32 |
| | AAACGGTACTCGTGACCACAAGAAAGGT | |
| N90100 | CGTTCTGATTATAAATTTTATGAAGATGA | SEQ ID NO:32 |
| | AAACGGTACTCGTGACCACAAGAAAGGT | |
| N10567RM | CGTTCTGATTATAAATTTTATGAAGCTGC | SEQ ID NO:33 |
| | AAACGGTACTCGTGACCACAAGAAAGGT | |
| N1060MEE | CGTTCTGATTATAAATTTTATGAAGCTGC | SEQ ID NO:33 |
| | AAACGGTACTCGTGACCACAAGAAAGGT | |
| N172NP | CGTTCTGATTATAAATTTTATGAAGCTGC | SEQ ID NO:33 |
| | AAACGGTACTCGTGACCACAAGAAAGGT | |
| N1199MEE | CGTTCTGATTATAAATTTTATGAAGCTGC | SEQ ID NO:34 |
| | AAATGGTACTCGTGACCACAAGAAAGGT | |
| N10568LM | CGTTCTGATTATAAATTTTATGAAGCTGC | SEQ ID NO:33 |
| | AAACGGTACTCGTGACCACAAGAAAGGT | |
| N90121RM | CGTTCTGATTATAAATTTTATGAAGCTGC | SEQ ID NO:33 |
| | AAACGGTACTCGTGACCACAAGAAAGGT | |
| N86027NP | CGTTCTGATTATAAATTTTATGAAGTTGC | SEQ ID NO:35 |
| | AAACGGTACTCGTGACCACAAGAAAGGT | |
| NTHI-486 | CGTTCTGATTATAAATTTTATGAAGTTGC | SEQ ID NO:35 |
| | AAACGGTACTCGTGACCACAAGAAAGGT | |
| N1712MEE | CGTTCTGATTATAAATTTTATGAAGTTGC | SEQ ID NO:35 |
| | AAACGGTACTCGTGACCACAAGAAAGGT | |
| NTHI-503 | CGTTCTGATTATAAATTTTATGAAGCTGC | SEQ ID NO:33 |
| | AAACGGTACTCGTGACCACAAGAAAGGT | |
| NTHI-476 | CGTTCTGATTATAAATTTTATGAAGAAGC | SEQ ID NO:36 |
| | AAACGGTACTCGTGACCACAAGAAAGGT | |
| N166NP | CGTTCTGATTATAAATTTTATAATGATGC | SEQ ID NO:37 |
| | AAACGGTACTCGTGACCACAAGAAAAGT | |
| N1182MEE | CGTTCTGATTATAAATTTTATAATGATGC | SEQ ID NO:37 |
| | AAACGGTACTCGTGACCACAAGAAAAGT | |
| N1848NP | CGTTCTGATTATAAATTTTATGAAGTTGC | SEQ ID NO:38 |
| | AAACGGTACTCGTGACCACAAGAAAAGT | |
| N1371MEE | CGTTCTGATTATAAATTTTATGAAGTTGC | SEQ ID NO:38 |
| | AAACGGTACTCGTGACCACAAGAAAAGT | |
| NTHI-498 | CGTTCTGATTATAAATTTTATGAAGTTGC | SEQ ID NO:38 |
| | AAACGGTACTCGTGACCACAAGAAAAGT | |
| NTHI-606 | CGTTCTGATTATAAATTTTATGAAGTTGC | SEQ ID NO:38 |
| | AAACGGTACTCGTGACCACAAGAAAAGT | |
| N1848L | CGTTCTGATTATAAATTTTATGAAGTTGC | SEQ ID NO:38 |
| | AAACGGTACTCGTGACCACAAGAAAAGT | |
| NTHI-567 | CGTTCTGATTATAAATTTTATGAAGATGC | SEQ ID NO:39 |
| | AAACGGTACTCGTGACCGCAAGACAGGT | |
| NTHI-484 | CGTTCTGATTATAAATTTTATGAAGATGC | SEQ ID NO:40 |
| | AAACGGTACTCGTAAGCACAAGGAAGGT | |
| N10559RM | CGTTCTGATTATAAACTTTATGAAGTTGC | SEQ ID NO:41 |
| | AAACGGTACTCGTGACCACAAGAAAAGT | |
| NTHI-601 | CGTTCTGATTATAAATTTTATGAAGTTGC | SEQ ID NO:42 |
| | AAACGGTACTCGTGACCACAAGCAAAGT | |
| NTHI-481 | CGTTCTGATTATAAATTTTATGAAGTTGC | SEQ ID NO:42 |
| | AAACGGTACTCGTGACCACAAGCAAAGT | |
| NTHI-482 | CGTTCTGATTATAAATTTTATGAAGTTGC | SEQ ID NO:42 |
| | AAACGGTACTCGTGACCACAAGCAAAGT | |
| N1370MEE | CGTTCTGATTATAAATTTTATGAAGTTGC | SEQ ID NO:42 |
| | AAACGGTACTCGTGACCACAAGCAAAGT | |
| N226NP | CGTTCTGATTATAAATTTTATGAAGAAGC | SEQ ID NO:43 |
| | AAACGGTACTCGTGACCACAAGAGAAGT | |
| NTHI-480 | CGTTCTGATTATAAATTTTATGAAGATGC | SEQ ID NO:44 |
| | AAACGGTACTCGTGAGCGCAAGAGAGGT | |
| N1657MEE | CGTTCTGATTATAAATTTTATGAAGTTGC | SEQ ID NO:45 |
| | AAACGGTACTCGTGAGCGCAAGAAAGGT | |
| N267NP | CGTTCTGATTATAAATTTTATGAAGTTGC | SEQ ID NO:45 |
| | AAACGGTACTCGTGAGCGCAAGAAAGGT | |
| NTHI-490 | CGTTCTGATTATAAATTTTATGAAGTTGC | SEQ ID NO:45 |
| | AAACGGTACTCGTGAGCGCAAGAAAGGT | |
| NTHI-494 | CGTTCTGATTATAAATTTTATGAAGTTGC | SEQ ID NO:45 |
| | AAACGGTACTCGTGAGCGCAAGAAAGGT | |
| N214NP | CGTTCTGATTATAAATTTTATGAAGTTCC | SEQ ID NO:46 |
| | AAACGGTACTCGTGACCACAAGCAAAGT | |
| N250NP | CGTTCTGATTATAAACGTTATGAAGAAGC | SEQ ID NO:47 |
| | AAACGGTACTCGTAACCACGACAAAGGT | |
| N1521 | CGTTCTGATTATAAACGTTATGAAGAAGC | SEQ ID NO:47 |
| | AAACGGTACTCGTAACCACGACAAAGGT | |
| NTHI-605 | CGTTCTGATTATAAACGTTATGAAGAAGC | SEQ ID NO:47 |
| | AAACGGTACTCGTAACCACGACAAAGGT | |
| NTHI-499 | CGTTCTGATTATGAATTTTATGAAGCTCC | SEQ ID NO:48 |
| | AAACAGTACTCGTGACCACAAGAAAGGT | |

TABLE 7
Cumulated Group 2 Gene Sequences

| | | |
|---|---|---|
| N1715MEE | CGTTCTGACTATAAATTGTACAATAAAAA | SEQ ID NO:49 |
| | TAGTAGTAGTAATAGTACTCTTAAAAACC | |
| | TAGGCGAA | |
| N1714MEE | CGTTCTGACTATAAATTGTACAATAAAAA | SEQ ID NO:49 |
| | TAGTAGTAGTAATAGTACTCTTAAAAACC | |
| | TAGGCGAA | |
| N86027RM | CGTTCTGACTATAAATTGTACAATAAAAA | SEQ ID NO:49 |
| | TAGTAGTAGTAATAGTACTCTTAAAAACC | |
| | TAGGCGAA | |
| N297NP | CGTTCTGACTATAAATTGTACAATAAAAA | SEQ ID NO:49 |
| | TAGTAGTAGTAATAGTACTCTTAAAAACC | |
| | TAGGCGAA | |
| N266NP | CGTTCTGACTATAAATTGTACAATAAAAA | SEQ ID NO:49 |
| | TAGTAGTAGTAATAGTACTCTTAAAAACC | |
| | TAGGCGAA | |
| N1885MEE | CGTTCTGACTATAAATTGTACAATAAAAA | SEQ ID NO:49 |
| | TAGTAGTAGTAATAGTACTCTTAAAAACC | |
| | TAGGCGAA | |
| NTHI-546 | CGTTCTGACTATAAATTGTACAATAAAAA | SEQ ID NO:49 |
| | TAGTAGTAGTAATAGTACTCTTAAAAACC | |
| | TAGGCGAA | |
| NTHI-604 | CGTTCTGACTATAAATTGTACAATAAAAA | SEQ ID NO:49 |
| | TAGTAGTAGTAATAGTACTCTTAAAAACC | |
| | TAGGCGAA | |
| NTHI-492 | CGTTCTGACTATAAATTGTACAATAAAAA | SEQ ID NO:50 |
| | TAGTAGT---AATAGTACTCTTAAAAACC | |
| | TAGGCGAA | |
| NTHI-502 | CGTTCTGACTATAAATTGTACGATAAAAA | SEQ ID NO:51 |
| | TAGTAGTAGTAAT---ACTCTTAAAAAAC | |
| | TAGGCGAA | |
| NTHI-506 | CGTTCTGACTATAAATTGTACAATAAAAA | SEQ ID NO:50 |
| | TAGTAGT---AATAGTACTCTTAAAAACC | |
| | TAGGCGAA | |
| N1236MEE | CGTTCTGACTATAAATTGTACAATAAAAA | SEQ ID NO:52 |
| | TAGTAGT---------ACTCTTAAAGACC | |
| | TAGGCGAA | |
| NTHI-500 | CGTTCTGACTATAAATTGTACAATAAAAA | SEQ ID NO:52 |
| | TAGTAGT---------ACTCTTAAAGACC | |
| | TAGGCGAA | |
| NTHI-183 | CGTTCTGACTATAAATTGTACAATAAAAA | SEQ ID NO:52 |
| | TAGTAGT---------ACTCTTAAAGACC | |
| | TAGGCGAA | |
| N165NP | CGTTCTGACTATAAATTGTACAATAAAAA | SEQ ID NO:53 |
| | TAGTAGTAAT------ACTCTTAAAGACC | |
| | TAGGCGAA | |
| NTHI-495 | CGTTCTGACTATAAATTATACAATAAAAA | SEQ ID NO:54 |
| | TAGTAGTGAT------GCTCTTAAAAAAC | |
| | TAGGCGAA | |

TABLE 8
Cumulated Group 3 Gene Sequences

| | | |
|---|---|---|
| N1729MEE | CGTTCTGACTATAAATTCTACGATAATAA | SEQ ID NO:59 |
| | ACGCATCGAT | |
| NTHI-504 | CGTTCTGACTATAAATTCTACGATAATAA | SEQ ID NO:59 |
| | ACGCATCGAT | |
| NTHI-544 | CGTTCTGACTATAAATTCTACGATAATAA | SEQ ID NO:59 |
| | ACGCATCGAT | |
| NTHI-600 | CGTTCTGACTATAAATTCTACGATAATAA | SEQ ID NO:59 |
| | ACGCATCGAT | |
| N1728MEE | CGTTCTGACTATAAATTCTACGATAATAA | SEQ ID NO:59 |
| | ACGCATCGAT | |
| N10548LM | CGTTCTGACTATAAATTCTACGATAATAA | SEQ ID NO:59 |
| | ACGCATCGAT | |
| N10547RM | CGTTCTGACTATAAATTCTACGATAATAA | SEQ ID NO:59 |
| | ACGCATCGAT | |
| N105678R | CGTTCTGACTATAAATTCTACGATAATAA | SEQ ID NO:59 |
| | ACGCATCGAT | |

TABLE 9
Cumulated Group 1, 2a, 2b, and 3 Gene Sequences

| | | |
|---|---|---|
| N1128 | CGTTCTGATTATAAATTTTATGAAGATGC | SEQ ID NO:29 |
| | AAACGGTACTCGTGACCACAAGAAAGGT | |
| N1715MEE | CGTTCTGACTATAAATTGTACAATAAAAA | SEQ ID NO:49 |
| | TAGTAGTAGTAATAGTACTCTTAAAAACC | |
| | TAGGCGAA | |
| NTHI-183 | CGTTCTGACTATAAATTGTACAATAAAAA | SEQ ID NO:52 |
| | TAGTAGT---------ACTCTTAAAGACC | |
| | TAGGCGAA | |
| N1729MEE | CGTTCTGACTATAAATTCTACGATAAT-- | SEQ ID NO:55 |
| | ----------------AAACGCATCGAT | |

The study shows that the LB1 (f) peptides of the P5-like fimbrin protein from all 83 ntHi isolates tested can be classified in three groups, and that both United States and European ntHi isolates fall into this classification.

### Example 2

### The Expression of LPD-LB1(f) peptide fusion polypeptides in E. coli

Source Material

1) The expression vector pMG1

The expression vector pMG1 is a derivative of pBR322 in which bacteriophage λ derived control elements for transcription and translation of foreign inserted genes were introduced (Young et al. (1983) PNAS USA 80, 6105-6109). In addition, the Ampicillin resistance gene was exchanged with the Kanamycin resistance gene.

The vector contains the λ promoter P_{L}, operator O_{L} and two utilization sites (Nut_{L} and NUt_{R}) to relieve transcriptional polarity effects. Vectors containing the P_{L} promoter, are introduced into an E. Coli lysogenic host to stabilize the plasmid DNA. Lysogenic host strains contain replication-defective λ phage DNA integrated into the genome. The chromosomal λ phage DNA directs the synthesis of the cI repressor protein which binds to the O_{L} repressor of the vector and prevents binding of RNA polymerase to the P_{L} promoter and thereby transcription of the inserted gene. The cI gene of the expression strain AR58 contains a temperature sensitive mutant so that P_{L} directed transcription can be regulated by temperature shift, i.e. an increase in culture temperature inactivates the repressor and synthesis of the foreign protein is initiated. This expression system allows controlled synthesis of foreign proteins especially of those that may be toxic to the cell.

2) The Expression Vector pMGMCS

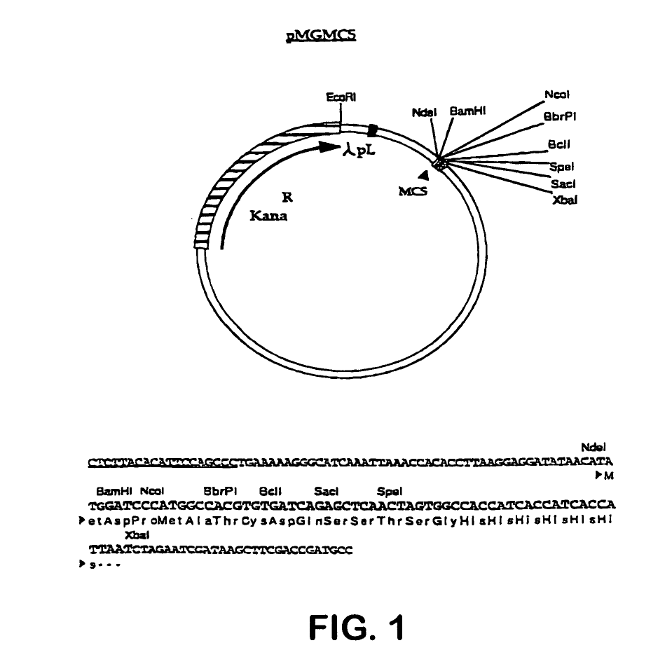

The nucleotide sequence between the BamHI and the XbaI restriction sites in pMG1 was replaced by a multiple cloning site DNA fragment (MCS) to generate the pMGMCS expression vector (FIG. 1).

A poly-His sequence has been added at the 3′ end of the MCS sequence to allow the expression of a protein product fused to a 6-Histidine tail.

The sequence coding for the first 3 amino acids of NS1 (Met-Asp-Pro) is present on the vector, before the BamHI restriction site.

3) Construction of Vector pRIT14588

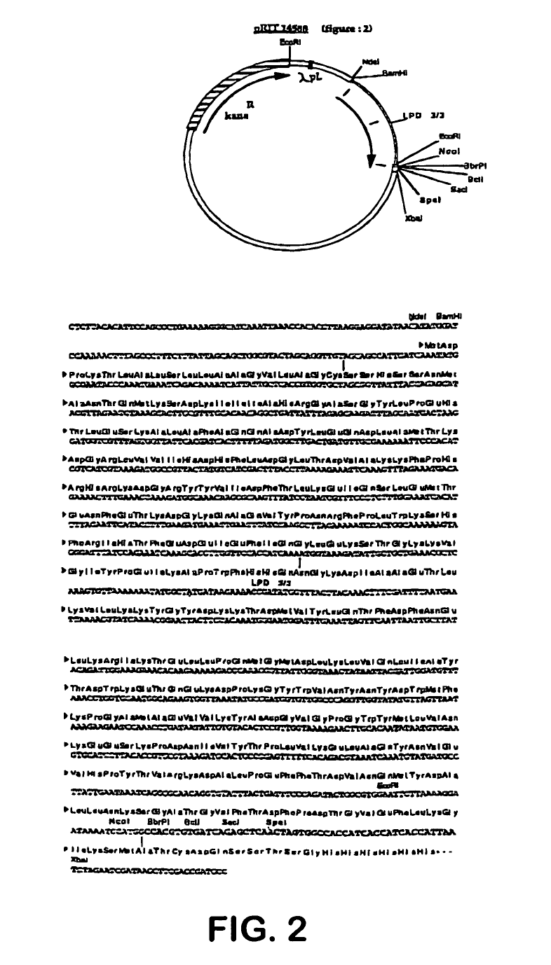

The cloning strategy for the generation of the pRIT14588 expression vector from the pMGMCS vector is outlined in FIG. 2. The lipoprotein D gene was amplified by PCR from the pHIC348 vector (Janson et al. (1991) Infect. Immun. 59, 119-125) with PCR primers containing BamHI and NcoI restriction sites at the 5′ and 3′ ends, respectively. The BamHI/NcoI fragment was then introduced into pMGMCS between BamHI and NcoI.

The lipoprotein D gene product contains its native signal sequence except for the first three amino acids which have been replaced by Met-Asp-Pro from NS1.

pRIT14588 was used to introduce LB1(f) peptides to the 3′ end of the Lipoprotein D gene. The LB1(f) peptides used were the following: group 1, ntHi-1128 (SEQ ID NO:5); group 2, ntHi-1715 MEE (SEQ ID NO: 2); group 3, ntHi-1729 MEE (SEQ ID NO: 3).

4) The E. coli Strain AR58

The AR58 lysogenic E. coli strain used for the production of the protein D carrier protein is a derivative of the standard NIH E. coli K12 strain N99 (F-su-galK2, lacZ⁻thr⁻). It contains a defective lysogenic λ phage (galE::TN10, λ Kil⁻ cI857 DH1). The Kil⁻ phenotype prevents the shut down of host macromolecular synthesis. The cI857 mutation confers a temperature sensitive lesion to the cI repressor. The DH1 deletion removes the λ phage right operon and the hosts bio, uvr3, and chlA loci. The AR58 strain (Mott et al. (1985) PNAS USA. 82, 88-92) was generated by transduction of N99 with a P1 phage stock previously grown on an SA500 derivative (galE::TN10, λ Kil⁻ cI857 DH1). The introduction of the defective lysogen into N99 was selected with tetracycline (a TN10 transposon coding for tetracyclin resistance is present in the adjacent galE gene).

### Example 2a

### Producing a Lipoprotein D-LB1 (f) Group 1 Fusion

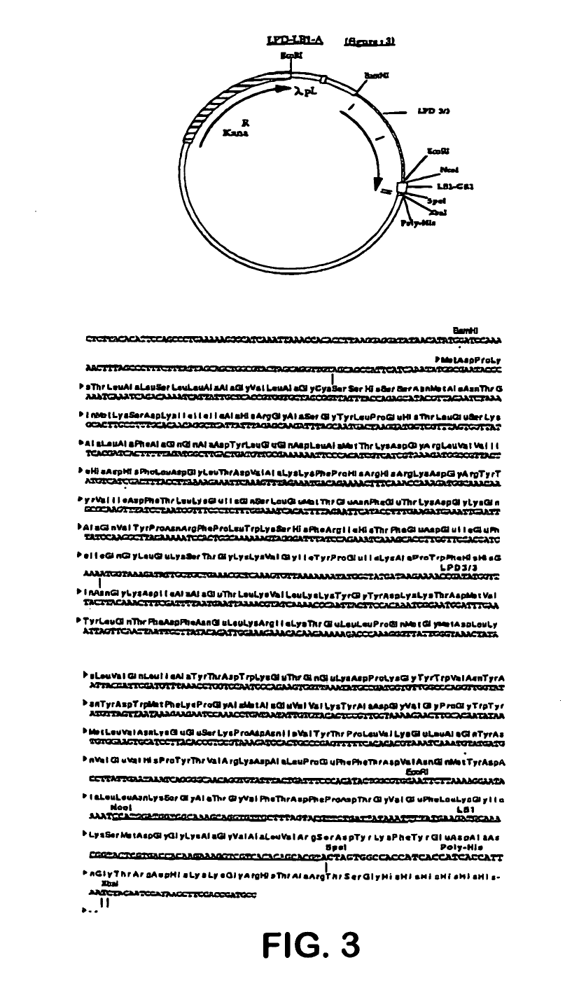

The aim of this construct was to clone the 19 residue LB1 (f) peptide 3′ to the NcoI site of the multiple cloning site of pRIT14588. Immediately 3′ to the NcoI site, two Glycine residues were introduced to place the LB1 (f) peptide gene in frame with the LPD gene. After the two Gly residues, the DNA coding for 8 natural residues N-terminal to the LB1(f) peptide (from the P5-like fimbrin protein) were introduced followed by the LB1(f) DNA sequence, followed by the DNA coding for the 5 natural residues C-terminal to the LB1(f) peptide. The plasmid (called LPD-LB1-A) is shown in FIG. 3 and was made as follows:

pRIT 14588 was cleaved with NcoI and SpeI, and the linear large fragment was dephosphorylated. The LB1(f) peptide gene was amplified up from the ntHi-1128 P5-like fimbrin gene with the following primers:

Primer LB-Baka-01 (5′-containing an NcoI site)

| | |
|---|---|
| 5′-CTA-GCC-ATG-GAT-GGT-GGC-AAA-GCA- | (SEQ ID NO:67) |
| GGT-G-3′ | |

Primer LB-Baka-05 (3′-containing an SpeI site)

| | |
|---|---|
| 5′-CAC-TAG-TAC-GTG-CGT-TGT-GAC-GAC- | (SEQ ID NO:68) |
| 3′ | |

The DNA produced by PCR amplification was cleaved with NcoI and SpeI. The LB1(f) DNA fragment was purified, and ligated into the NcoI and SpeI sites of the cleaved pRIT14588. The ligation mixture was transformed into E. coli AR58, and the transformation product was spread onto solid medium (BP) LBT+Kanamycin (50 μg/mL). The plates were incubated at 30° C. overnight. Transformants were checked by PCR, and positive candidates were grown in liquid culture at 30° C. In order to initiate expression of the LPD-LB1(f) chimeric polypeptide, the culture was subjected to a change in temperature from 30° C. to 39° C. during 4 hours. Expression was monitored on a 12.5% acrylamide gel (viewed either with Coomassie stain and/or Western Blot). The molecular size of the chimeric polypeptide was about 44 kDa.

### Example 2b

### Producing a LPD-LB1(f) Group 2+LB1(f) Group 1 fusion

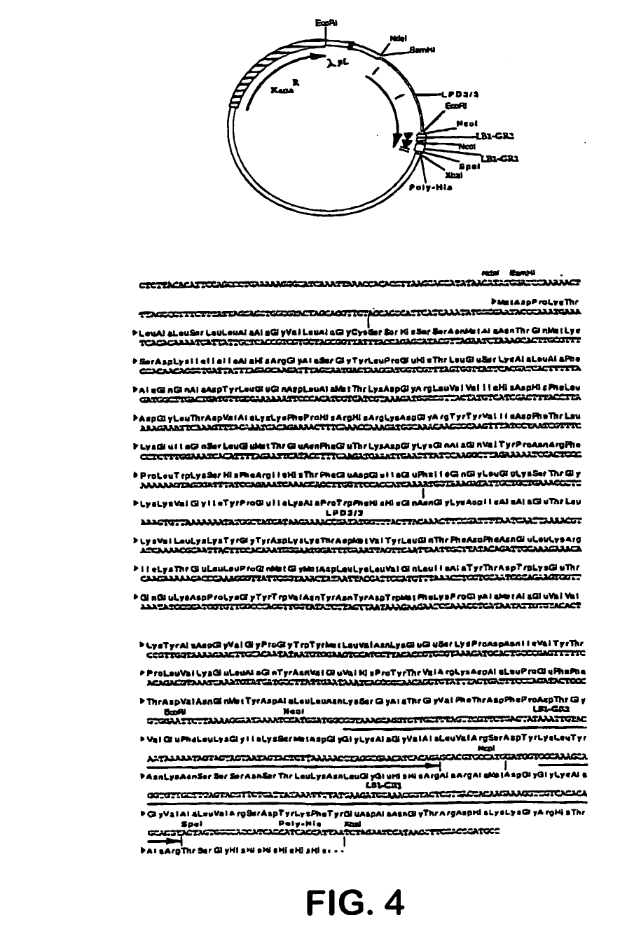

The plasmid (called LPD-LB1-II) is shown in FIG. 4 and was made as follows:

Plasmid LPD-LB1-A was cleaved with NcoI and the linear DNA was dephosphorylated. The Group 2 LB1(f) peptide gene was amplified up from the ntHi-1715MEE P5-like fimbrin gene with the following primers:

Primer NT1715-11NCO (5′ containing an NcoI site)

| | |
|---|---|
| 5′-CAT-GCC-ATG-GAT-GGC-GGT-AAA-GCA- | (SEQ ID NO:69) |
| GGT-GTT-GCT-3′ | |

Primer NT1715-12NCO (3′ containing an NcoI site)

| | |
|---|---|
| 5′-CAT-GCC-ATG-GCA-CGT-GCT-CTG-TGA- | (SEQ ID NO:70) |
| TG-3′ | |

The DNA produced by PCR amplification was cleaved with NcoI. The LB1(f) DNA fragment was purified, and ligated into the open NcoI site of the cleaved LPD-LB1-A plasmid (5′ to the gene for the Group 1 LB1(f) peptide). The ligation mixture was transformed into E. coli AR58, and the transformation product was spread onto solid medium (BP) LBT+Kanamycin (50 μg/mL). The plates were incubated at 30° C. overnight. Transformants were checked by PCR, and positive candidates were grown in liquid culture at 30° C. In order to initiate expression of the LPD-LB1(f)_{2,1} chimeric polypeptide, the culture was subjected to a change in temperature from 30° C. to 39° C. during 4 hours. Expression was monitored on a 12.5% acrylamide gel (viewed either with Coomassie stain and/or Western Blot). The molecular size of the chimeric polypeptide was about 50 kDa.

### Example 2c

### Producing a Lipoprotein D-LB1(f) Group 2+LB1(f) Group 1+LB1(f) Group 3 fusion

The plasmid (called LPD-LB1-III) is shown in FIG. 5 and was made as follows:

Plasmid LPD-LB1-II was cleaved with SpeI and the linear DNA was dephosphorylated. The Group 3 LB1(f) peptide gene from ntHi-1929MEE was made by hybridising the following primers:

Primer NT1729-18 SPE (5′-containing a cleaved SpeI
site at 5′ end)

| | |
|---|---|
| 5′-CTA-GTC-GTT-CTG-ACT-ATA-AAT-TCT- | (SEQ ID NO:71) |
| ACG-ATA-ATA-AAC-GCA-TCG-ATA-GTA-3′ | |

Primer NT1729-19 SPE (3′-containing a cleaved SpeI
site at 3′ end)

| | |
|---|---|
| 5′-CTA-GTA-CTA-TCG-ATG-CGT-TTA-TCG- | (SEQ ID NO:72) |
| TAG-AAT-TTA-TAG-GCA-GAA-CGA 3′ | |

The hybridised DNA contained the gene for the Group 3 LB1(f) peptide and a cleaved SpeI at either end. The LB1(f) DNA fragment was ligated into the open SpeI site of the cleaved LPD-LB1-II plasmid (3′ to the gene for the Group 1 LB1(f) peptide). The ligation mixture was transformed into E. coli AR58, and the transformation product was spread onto solid medium (BP) LBT+Kanamycin (50 μg/mL). The plates were incubated at 30° C. overnight. Transformants were checked by PCR, and positive candidates were grown in liquid culture at 30° C. In order to initiate expression of the LPD-LB1(f)_{2,1,3} chimeric polypeptide, the culture was subjected to a change in temperature from 30° C. to 39° C. during 4 hours. Expression was monitored on a 12.5% acrylamide gel (viewed either with Coomassie stain and/or Western Blot). The molecular size of the chimeric polypeptide was about 53 kDa.

### Example 2d

### Characterisation of the Expression of the Chimeric Polypeptides

Expression of the above chimeric polypeptides was monitored on a 12.5% acrylamide gel which was observed as either:

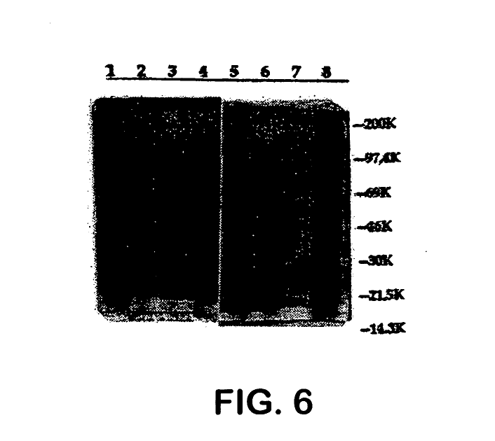

a) a Coomassie stained gel (FIG. 6)

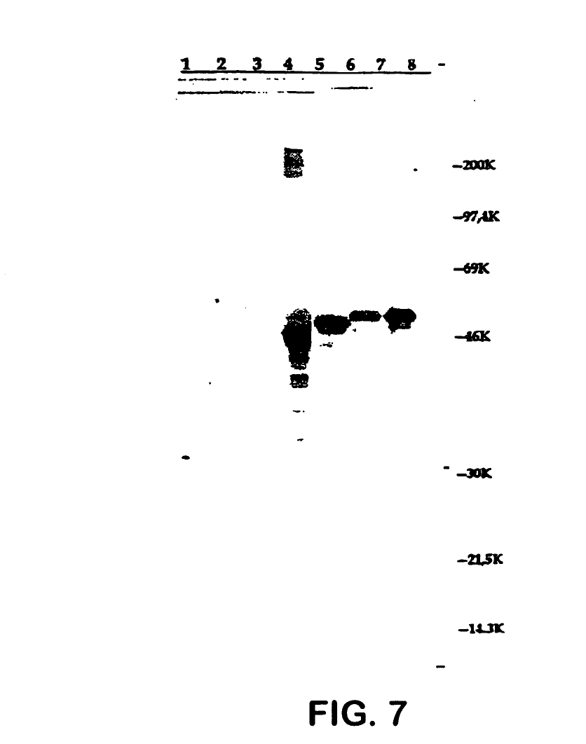

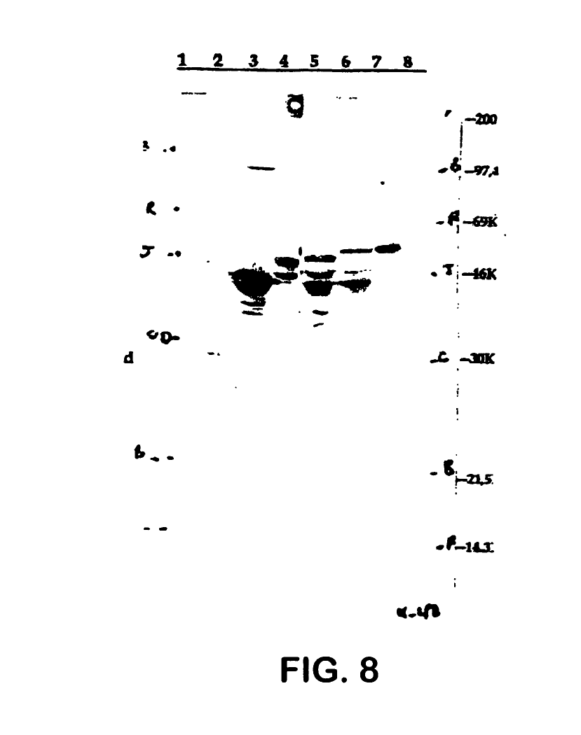

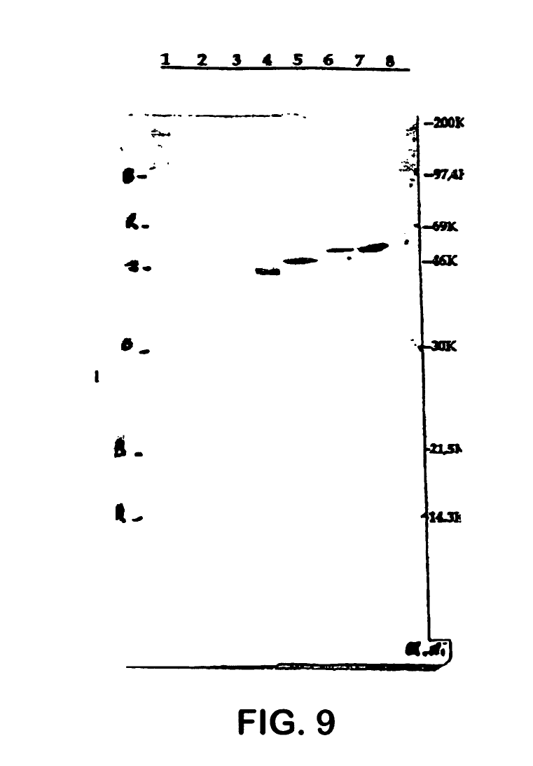

b) a Western blot
1) using rabbit anti-LB1 antibodies (FIG. 7)
2) using a monoclonal anti-LPD antibody (FIG. 8)
3) using an antibody against the six-Histidine Purification Tag (FIG. 9)

As can be observed, each chimeric polypeptide can be expressed efficiently in E. coli.

### Example 3

### Purification of the Chimeric Polypeptides

The purification of LPD-LB1(f)_{2,1,3} (expressed using the construct shown in FIG. 5) was achieved as follows.

The E. coli were washed and resuspended in phosphate buffer (50 mM, pH 7.0). The cells were lysed by gently swirling them overnight at 4° C. in the presence of 3% Empigen. The solution was then centrifuged for 30 minutes at 8,000 rpm in a Beckman JA10 rotor. The supernatant was diluted 4 times in 50 mM phosphate buffer, 500 mM NaCl, pH 7.0. The first stage of purification was achieved on a Qiagen NTA Ni++ column due to the presence of the six histidine tag at the C-terminus of the polypeptide. The column was equilibrated with 10 mM sodium phosphate buffer, 500 mM NaCl, 0.5% Empigen, pH7.5, and the polypeptide was eluted off the column with an imidazole gradient (0-100 mM) in 20 mM sodium phosphate buffer, 0.5% Empigen, pH7.0. Elution was followed by running fractions on SDS-PAGE gels.

The next step in the purification was on a Bio-Rad Macro-Prep 50S column. The polypeptide bound to the column equilibrated in 20 mM phosphate buffer, 0.5% Empigen, pH 7.0, and was eluted from the column using a gradient of 0 to 500 mM NaCl in the same buffer. Elution was followed by running fractions on SDS-PAGE gels.

The last (polishing) step of the process was done using a Sephacryl S200 HR size exclusion column. The polypeptide solution from the previous step was firstly concentrated with a Filtron Omega 10 kDa concentrator device. The resulting solution was loaded and run on the column equilibrated with PBS buffer with 0.5% Empigen. Elution of the polypeptide was followed by running fractions on SDS-PAGE gels.

The pooled fractions were filtered through a 0.22 μm filter. The resulting protein runs as one pure band on a Coomassie stained SDS-PAGE gel, and the equivalent Western blot using an anti-LB1 antibody. Tests showed that the protein remained intact even after 7 days at 37° C.

Approximately 200 mg of polypeptide per litre of cell culture can be purified by this method.

### Example 4

### Preclinical Experimentation on Vaccine Effectiveness of the Chimeric Polypeptides

### Example 4a

### Generation of Antisera

Antisera was generated against 4 types of antigen: LPD; PD; LPD-LB1(f)_{2,1,3} (made recombinantly using plasmid LPD-LB1-III); LB1 (a group 1 LB1(f) peptide fused to a T-cell promiscuous epitope from measles virus fusion protein, the sequence of the peptide being: RSDYKFYEDANGTRDHKKGPSLKLLSLIKGVIVHRLEGVE) (SEQ ID NO:28).

Four cohorts comprising 5 chinchillas were immunised, each cohort with one of the immunogens identified above. The dosage was 10 μg antigen/200 μL AlPO₄/20 μg MPL (3-O-deacylated monophosphoryl lipid A) for the first three antigens, and 10 μg antigen delivered in Complete or Incomplete Freund's Adjuvant (CFA or IFA) for LB1.

A total of three doses were injected at one month intervals. Fifteen days after the final immunization, all animals were bled by cardiac puncture and thorectomy for collection of serum. Serum was pooled by cohort and stored at −70° C.

Titres obtained were 10-50K for anti-PD serum, 50K for anti-LPD, 50-100K for anti-LB1 and 50-100K for anti-LPD-LB1(f)_{2,1,3}. In addition to the LB1 peptide, anti-LB1 recognised LPD-LB1(f)_{2,1,3} on a Western blot. Anti-LPD and anti-PD also recognised LPD-LB1(f)_{2,1,3}. Immunogold labeling experiments (using gold-conjugated protein A) showed that anti-LB1 & anti-LPD-LB1(f)_{2,1,3} polyclonal antisera both recognized surface accessible epitopes on ntHi 86-028NP cells similar to those recognised by a monoclonal antibody against the p5-like fimbrin protein.

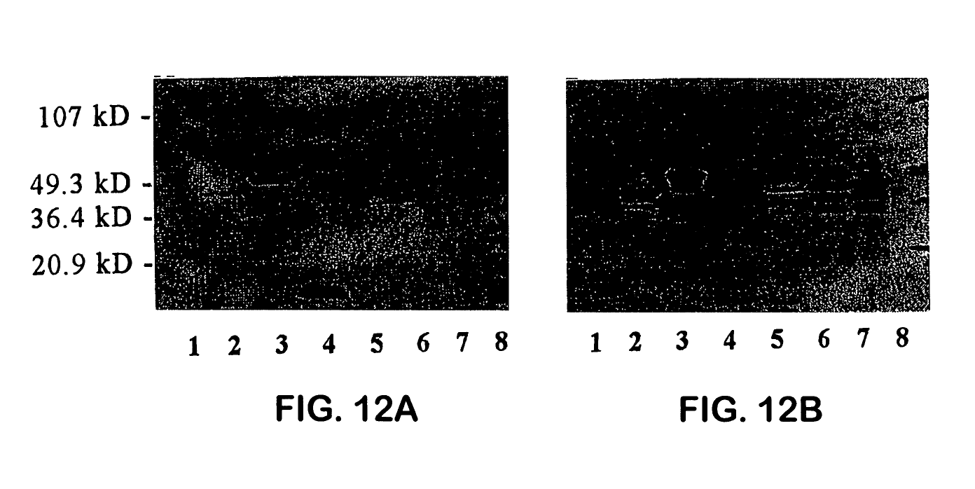

In addition, FIG. 12 shows a Western blot indicating that the anti-LPD-LB1(f)_{2,1,3} serum recognises the P5-like fimbrin protein from three ntHi strains representing the 3 major LB1(f) groups. The recognition of these strains by anti-LPD-LB1(f)_{2,1,3} is far stronger than by anti-LB1.

### Example 4b

### Passive Transfer and Challenge

This study aimed to perform an in vivo challenge study of passively immunised chinchillas to determine the relative efficiency among the 4 immunogen (or sham) formulations to facilitate clearance of ntHi from the nasopharynx.

Five cohorts of 11 chinchillas each (Chinchilla lanigera) free of middle ear disease were inoculated intranasally on day-7 with 6×10⁶ TCID₅₀ adenovirus type 1. On day-1 each chinchilla cohort was passively immunised with a 1:5 dilution of one of the four serum samples described in Example 4a via cardiac puncture. The fifth cohort (the sham) received pyrogen-free sterile saline solution by cardiac puncture instead. About 5 mL serum/kg animal was administered.

On day 0 the cohorts were intranasally challenged with ntHi: about 10⁸ cfu ntHi # 86-028NP (group 1) per animal. Statistical evaluation of the passive transfer study was performed prior to de-blinding the study.

This sequential inoculation with two pathogens closely mimics both the natural route of acquisition of these agents as well as their synergistic interaction in the human host.

The severity of the disease was scored by otoscopic observation. This is rated on a 0-4 scale. Signs of tympanic membrane (TM) inflammation were observed to obtain a score: the presence of effusion, small vessel dilation, air-fluid interface, opacity, etc.

A repeated measures analysis of variance was used to compare the pattern of responses over time (days) and ear (left or right) for the five groups (cohorts). Due to the large number of repeat observations on each animal, the analysis was divided into 5 sections as follows: days 1-7, days 8-14, days 19-21, days 22-28, and days 29-33. There was little variation in the responses on days-7 through 0, 34 and 35 and therefore no such analysis was performed on those times. Where possible (when there was non-zero variability in the mean response), tests were performed to compare the mean responses between the groups at these time points. Tukey's HSD test was used for all post-hoc multiple comparisons. Significance was assessed using an alpha level of 0.05.

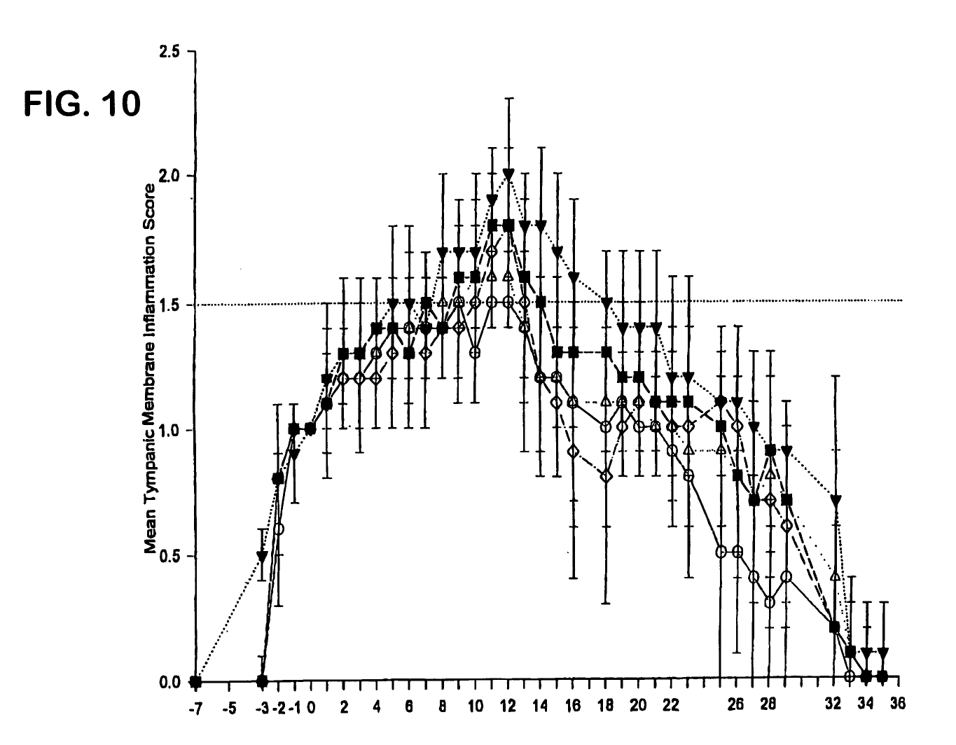

The results are shown in FIG. 10. Inflammation increases over time for all groups in a significant manner during the period of day 1 to 7. During days 29-33 inflammation decreased over time in a significant manner for all groups. As can be seen from the data, the serum containing antibodies against recombinant LPD-LB1(f)_{2,1,3} helped to reduce the TM inflammation throughout the experiment. An effective vaccinogen should maintain TM inflammation at or below 1.5 for the duration of the study period. LPD-LB1(f)_{2,1,3} anti-serum only allowed the mean inflammation score to rise above 1.5 for 2 days as well as inducing a consistent downward trend thereafter.

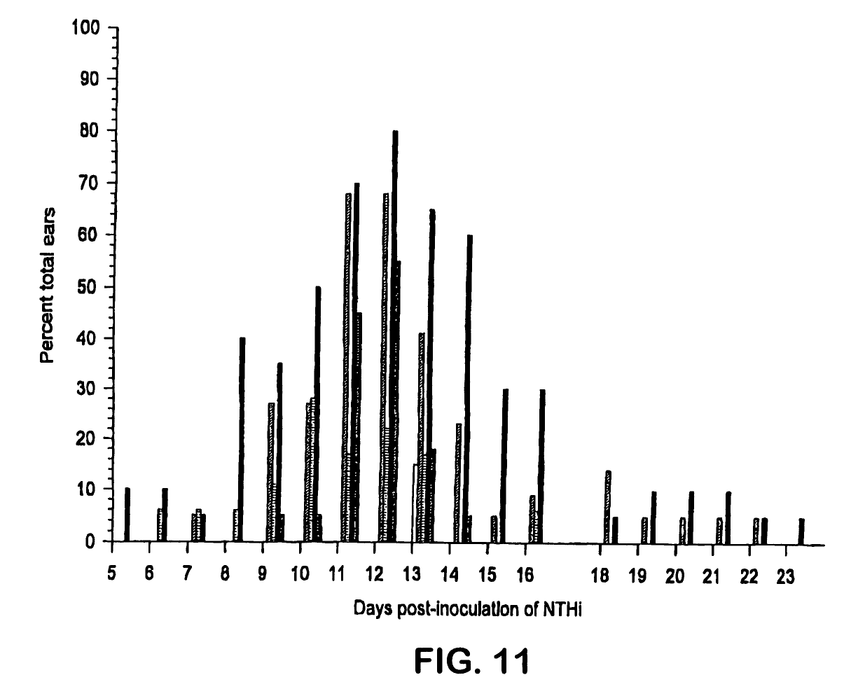

In addition to otoscopy, tympanometry (EarScan, South Daytona, Fla., USA), which measures changes in middle ear pressure, was also employed. These two measurements can be used in conjunction to give a reliable indication of whether an effusion has taken place in a middle ear. Tympanometry results indicated an abnormal ear if: a type B tympanogram was obtained, or middle ear pressure was less than −100 daPa. FIG. 11 shows the results of this analysis. Clearly, the recombinant LPD-LB1(f)_{2,1,3} performed well in this study when considering the outcome measures of preventing both TM inflammation and the development of effusion. Overall LPD-LB1(f)_{2,1,3} ranks second only to the positive control, the LB1 peptide. The LB1 peptide, however, was adjuvanted with CFA (a very strong adjuvant) and can therefore not be directly compared to the LPD-LB1(f)_{2,1,3} result.

A statistical evaluation on the data presented in FIG. 11 is presented in Table 10. The evaluation compared the reduction in percent effusion in each immunized cohort to that observed in sham immunized animals during peak incidence of disease [the four days of observation in which at least 50% of sham ears contained an effusion (had otitis media)].

The positive control (anti-LB1/CFA) was significant at p<0.001 on all four days (days 11-14). Anti-LPD-LB1(f)_{2,1,3} inhibited the development of otitis media at a p-value <or equal to 0.001 on days 11, 12, 13 and 14 also. Anti-PD was significant on days 13 and 14 only whereas anti-LPD was able to prevent the development of otitis media relative to sham animals on day 14 only (p value close to 0.02).

The recombinant LPD-LB1(f)_{2,1,3} polypeptide therefore significantly inhibits the development of otitis media in chinchillas which were passively transferred with this serum pool.

TABLE 10
A comparison of % ears containing effusion in the LB1, PD, LPD-
LB1(f)213, and LPD groups with % of ears containing effusion in the
Sham group on days 11 through 14..

| Day | Group | % Effusion | p-value |
|---|---|---|---|
| 11 | LB1 | 0 | <0.0001 |
| (Sham = 70%) | PD | 45 | 0.1010 |
| | LPD-LB1(f)213 | 17 | 0.0010 |
| | LPD | 68 | 0.8886 |
| 12 | LB1 | 0 | <0.0001 |
| (Sham = 80%) | PD | 55 | 0.0854 |
| | LPD-LB1(f)213 | 22 | 0.0004 |
| | LPD | 68 | 0.3788 |
| 13 | LB1 | 15 | 0.0012 |
| (Sham = 65%) | PD | 18 | 0.0020 |
| | LPD-LB1(f)213 | 17 | 0.0002 |
| | LPD | 41 | 0.1188 |
| 14 | LB1 | 0 | <0.0001 |
| (Sham = 60%) | PD | 5 | 0.0002 |
| | LPD-LB1(f)213 | 0 | <0.0001 |
| | LPD | 23 | 0.0146 |

### Example 4c

### Adherence Inhibition Data

An established single cell adherence assay was carried out using human oropharyngeal cells. The mean percent inhibition of adherence (±sem) of ntHi strains to these cells by the immune chinchilla sera produced in Example 4a. The results using anti-sera against LPD-LB1(f)_{2,1,3} and LPD can be seen in Table 11. The anti-sera against LPD-LB1(f)_{2,1,3} was seen to be effective at inhibiting adherence of Group 1 and Group 2 ntHi strains. It was also more effective against all the strains than anti-LPD serum was.

TABLE 11
The mean percent inhibition of adherence (±sem) of ntHi strains to
human oropharyngeal cells by immune chinchilla sera.

| | ntHi | | | | | | | |
|---|---|---|---|---|---|---|---|---|
| Cohort | strain | | Pooled Serum Dilution | | | | | |
| Name | (Group) | n | 1:25 | 1:50 | 1:100 | 1:200 | 1:400 | 1:800 |
| LPD/ | 86-028L | 3 | 29 ± 3 | 31 ± 4 | 13 ± 7 | 19 ± 8 | 12 ± 5 | 16 ± 7 |
| AlPO₄/ | (Group 1) | | | | | | | |
| MPL | 1128MEE | 2 | 0 ± 0 | 12 ± 12 | 8 ± 5 | 12 ± 1 | 8 ± 8 | 16 ± 1 |
| | (Group 1) | | | | | | | |
| | 266NP | 3 | 46 ± 9 | 38 ± 7 | 24 ± 13 | 24 ± 21 | 30 ± 16 | 28 ± 19 |
| | (Group 2a) | | | | | | | |
| LPD- | 86-028L | 3 | 32 ± 2 | 36 ± 1 | 38 ± 2 | 27 ± 3 | 3 ± 2 | 19 ± 3 |
| LB1(f)₂₁₃/ | (Group 1) | | | | | | | |
| AlPO₄/ | 1128MEE | 2 | 24 ± 14 | 23 ± 4 | 30 ± 7 | 13 ± 13 | 11 ± 11 | 12 ± 6 |
| MPL | (Group 1) | | | | | | | |
| | 266NP | 3 | 52 ± 10 | 43 ± 3 | 36 ± 7 | 13 ± 10 | 6 ± 9 | 14 ± 19 |
| | (Group 2a) | | | | | | | |

### Example 4d

### Passive Transfer and Challenge with Heterogeneous ntHi Strains

A similar study was carried out as described in Example 4b) above using ntHi strains from different LB1(f) group classifications to challenge the chinchilla adenovirus co-infection model.

A total of 132 juvenile (approx. 300 g) chinchillas (Chinchilla lanigera) with no evidence of middle ear infection by either otoscopy or tympanometry were used for 2 challenge studies using anti-LB1 and anti-LPD-LB1(f)_{2,1,3} antisera. Mean weight of chinchillas for the two challenge studies detailed below were: 296±38 g for 298±42 g respectively. Animals were rested 10 days upon arrival and were then bled nominally by cardiac puncture for collection of pre-immune serum, which was stored at −70° C. until use. Animals were rested a minimum of 7 days from collection of pre-immune serum until receiving adenovirus.

The ntHi used in these studies are limited passage clinical isolates cultured from the middle ears or nasopharynges of children who underwent tympanostomy and tube insertion for chronic otitis media with effusion at Columbus Children's Hospital [86-028NP (group 1), 1885MEE (group 2) and 1728MEE (group 3)]. All isolates were maintained frozen in skim milk plus 20% glycerol (v/v) until streaked onto chocolate agar and incubated at 37° C. for 18 hours in a humidified atmosphere containing 5% CO₂. Adenovirus serotype 1 was also recovered from a paediatric patient at Columbus Children's Hospital.

For both passive transfer studies, 66 juvenile chinchillas were used to establish six cohorts of eleven chinchillas each. Naïve chinchilla sera was collected from these animals and screened individually by Western blot for the presence of any significant pre-existing antibody titre prior to enrolment in the study. Experiments were conducted as for Example 4b) above. Two cohorts received the LB1 antiserum pool, two cohorts received the LPD-LB1(f)_{2,1,3} antiserum pool, and two cohorts received pyrogen free sterile saline. Observers knew neither the antiserum received nor which animals formed a cohort group.

Chinchillas were intranasally challenged by passive inhalation of approximately 108 CFU of: ntHi 86-028NP, or 1885MEE per animal (study A); or ntHi 86-028NP, or 1728MEE per animal (study B). Each of these three strains was chosen to represent a different sequence heterogeneous ntHi group relative to peptide LB1(f): group 1 strain NTHi 86-028NP; group 2 NTHi strain 1885MEE; and group 3 NTHi strain 1728MEE.

As in Example 4b), animals were blindly evaluated by otoscopy and tympanometry daily, or every 2 days, from the time of adenovirus inoculation until 35 days after NTHi challenge. Signs of tympanic membrane inflammation were rated on a 0 to 4+ordinal scale and tympanometry plots were used to monitor changes in both middle ear pressure, tympanic width and tympanic membrane compliance. Tympanometry results indicated an abnormal ear if: a type B tympanogram was obtained; compliance was <0.5 ml or >1.2 ml; middle ear pressure was less than −100 daPa; or tympanic width greater than 150 daPa.

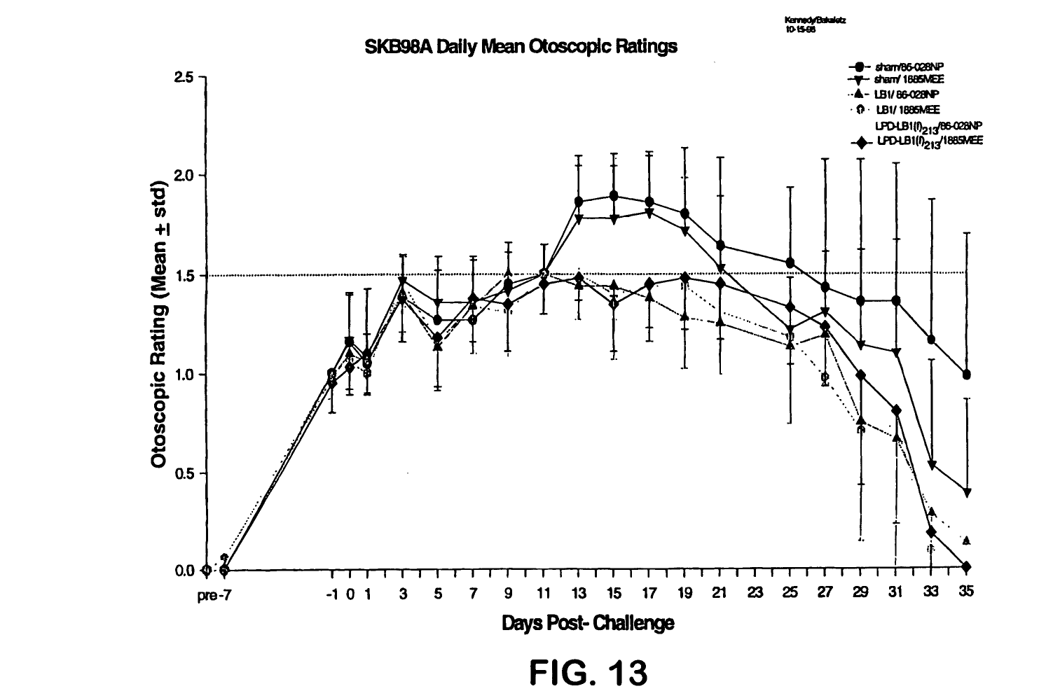

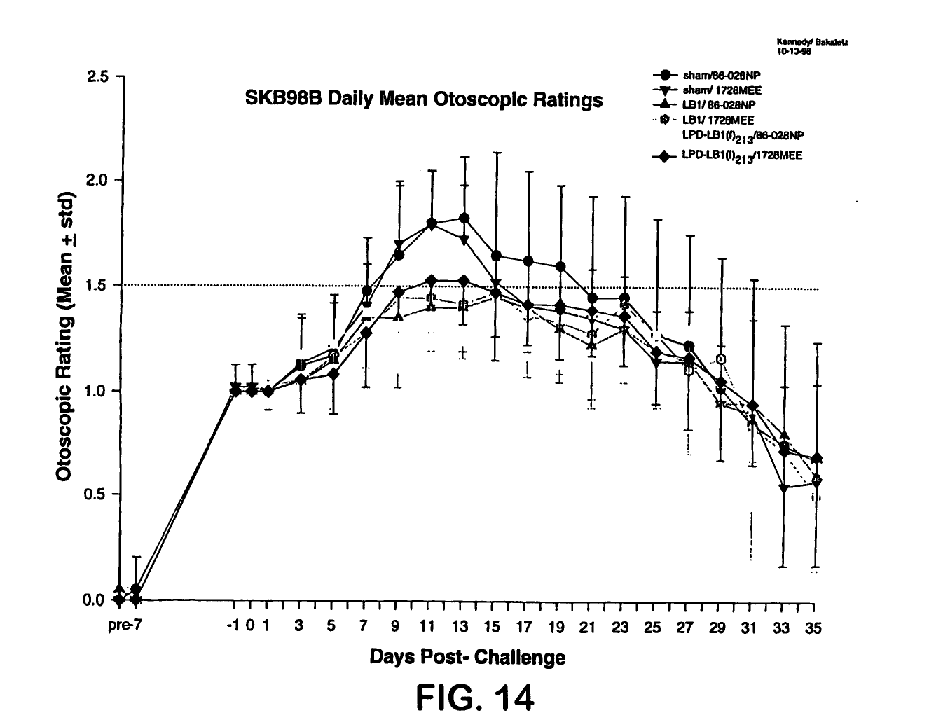

Tukey's HSD test was used to compare daily mean tympanic membrane inflammation scores among cohorts challenged with the same NTHi strain from day 1-35 after bacterial challenge. Each cohort of immunized animals had significantly lower mean otoscopy scores (p<0.05) than the sham cohort challenged with the same strain of NTHi for a minimum of 7 days (max. 22 days). Otoscopic rating results are shown in FIG. 13 (study A) and FIG. 14 (study B). The days on which the mean otoscopy scores were significantly less for LPD-LB1(f)_{2,1,3} than in the sham experiments were: days 13-35 (study A, 86-028NP); days 1-8, 12-21 (study A, 1885 MEE); days 8-14, 23 (study B 86-028NP); days 8-14 (study B, 1728 MEE).

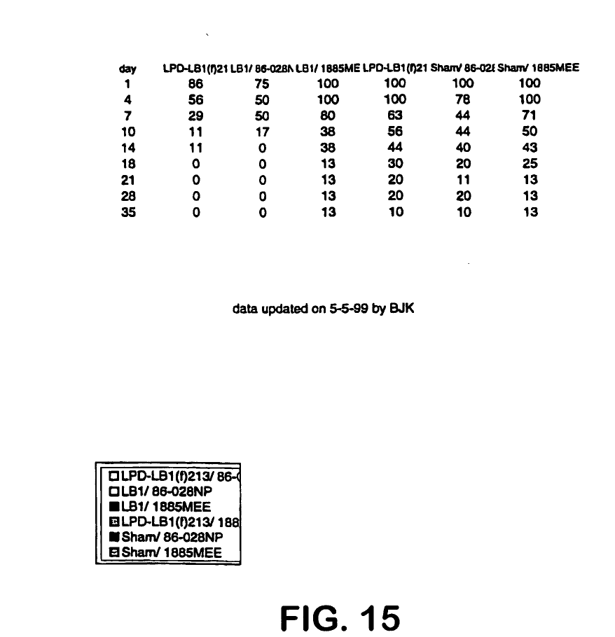

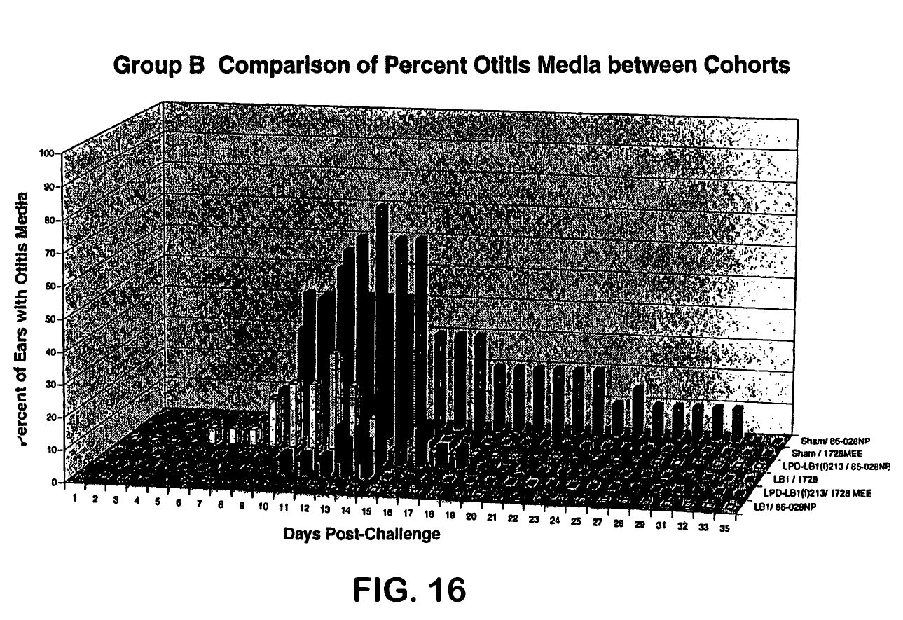

An analysis of the percentage of normal ears for studies A and B are shown in FIG. 15 and FIG. 16, respectively.

The ability of passive transfer of specific antisera to protect against the development of otitis media was assessed by a Z test. In both studies, animals which received anti-LB1 serum showed no signs of developing otitis media with effusion after challenge with NTHi 86-028NP. Days for which delivery of anti-LPD-LB1(f)_{2,1,3} serum significantly prevented the development of otitis media in comparison with sham animals (measured on days when greater than 50% of the sham animals had effusions) were: days 13-21 (study A, 86-028NP); days 13-18 (study A, 1885 MEE); days 13-14 (study B 86-028NP); days 9-12 (study B, 1728 MEE).

In summary, challenge of chinchillas with any of the three ntHi isolates used here resulted in initial colonization of the nasopharynx. Evaluation data obtained by otoscopy and tympanometry indicated that cohorts which received antiserum directed against LPD-LB1(f)_{2,1,3} had significantly lower mean otoscopy scores and a significant reduction in incidence of otitis media compared to sham cohorts challenged with the same strain of NTHi over many days of observation.

Thus, LPD-LB1(f)_{2,1,3} provided significant protection from the development of otitis media induced by heterologous strains of NTHi in adenovirus compromised chinchillas. In addition, LB1 also provided protection, however this may have been partly due to the strong adjuvant (CFA) used in conjunction with it.

Although certain embodiments of this invention have been shown and described, various adaptations and modifications can be made without departing from the scope of the invention as described in the appended claims. For example, peptides or polypeptides having the substantially the same amino acid sequence as described herein are within the scope of the invention.

We claim:

1-31. (canceled)

32. A peptide comprising one or more amino-acid sequences selected from the group consisting of:
SEQ ID No. 1,
SEQ ID No. 2,
SEQ ID No. 3, and
SEQ ID No. 4
or any antigenically related variants of said sequences which have an identity of at least 75% and are capable of immunologically mimicking the corresponding antigenic determinant site of the P5-like fimbrin protein of non-typeable Haemophilus influenzae, with the proviso that the antigenically related variants do not include those peptides provided in SEQ ID NO:5 or SEQ ID NO:6.

33. The peptide of claim 32 which comprises the amino-acid sequence provided in SEQ ID NO:1.

34. The peptide of claim 32 which comprises the amino-acid sequence provided in SEQ ID NO:2.

35. The peptide of claim 32 which comprises the amino-acid sequence provided in SEQ ID NO:3.

36. The peptide of claim 32 which comprises the amino-acid sequence provided in SEQ ID NO:4.

37. A chimeric polypeptide comprising one or more peptides of claim 32 covalently linked to a carrier polypeptide which comprises at least one T-cell epitope.

38. The chimeric polypeptide of claim 37 which also comprises a purification tag peptide sequence.

39. The chimeric polypeptide of claim 38 wherein the purification tag peptide sequence is a Histidine-tag sequence.

40. The chimeric polypeptide of claim 37 wherein the carrier polypeptide is lipoprotein D.

41. The chimeric polypeptide of claim 37 wherein the amino acid sequences of the polypeptides used are selected from the group consisting of SEQ ID NO: 1, 2, and 3.

42. A chimeric polypeptide comprising three LB1(f) subunits and lipoprotein D, wherein the amino acid sequences of the LB1 (f) subunits used are provided in SEQ ID NO: 2, 3, and 5.

43. The chimeric polypeptide of claim 42 which also comprises a Histidine purification tag sequence.

44. The chimeric polypeptide of claim 42 wherein the order of the peptide components from the N-terminus of the polypeptide is: lipoprotein D, LB1 (f) subunit (SEQ ID NO:2), LB1(f) subunit (SEQ ID NO:5), and LB1(f) subunit (SEQ ID NO:3).

45. The chimeric polypeptide of claim 44 wherein the amino acid sequence of the polypeptide is provided in FIG. 5.

46. A vaccine composition comprising an immunogenic amount of at least one peptide or polypeptide from claims 32-45 in a pharmaceutically acceptable excipient, and an optional adjuvant.

47. A method of inducing an immune response in a mammal susceptible to Haemophilis influenzae infection comprising the administration to the mammal of an effective amount of the vaccine according to claim 46.

48. A method of preventing Haemophilis influenzae infection comprising the administration to a mammal an effective amount of a vaccine according to claim 46.

49. A DNA or RNA molecule encoding one of the LB1(f) peptides or polypeptides provided in claims 32-45.

50. The DNA or RNA molecule of claim 49 wherein the DNA sequence of said LB1(f) polypeptide is provided in FIG. 5.

51. The DNA or RNA molecule of claim 47 contained within an expression vector, wherein said expression vector is capable of producing said LB1 (f) peptide or polypeptide when present in a compatible cell host.

52. A host cell comprising the expression vector of claim 49.

53. A process for producing a LB1(f) peptide or polypeptide comprising culturing the host cell of claim 50 under conditions sufficient for the production of said polypeptide and recovering the LB1 (f) peptide or polypeptide.

54. A process for producing LB1(f) peptide or polypeptide of claim 51 wherein the process comprises the steps of lysing the host cells, and purifying the soluble extract using an immobilised Nickel column step, a cation exchange column step, and a size exclusion column step.

55. A process for producing a host cell which produces a LB1(f) peptide or polypeptide thereof comprising transforming or transfecting a host cell with the expression vector of claim 49 such that the host cell, under appropriate culture conditions, expresses a LB1(f) peptide or polypeptide.

56. A purified antibody which is immunospecific to a peptide provided in claims 32-36.

57. A purified antibody which is immunospecific to a chimeric polypeptide provided in claims 37-45.

58. A method of detecting the presence of Haemophilus influenzae in a sample by contacting said sample with the antibody of claim 54 in the presence of an indicator.

59. A method of detecting the presence of Haemophilus influenzae in a sample by contacting said sample with a DNA probe or primer constructed to correspond to the wild-type nucleic acid sequence which codes for a LB1 (f) peptide of the P5-like fimbrin protein of Haemophilus influenzae, characterised in that the probe is selected from the group consisting of gene sequences as provided in Tables 6-8.

60. A reagent kit for diagnosing infection with Haemophilus inflenzae in a mammal comprising the DNA probes of claim 57.

61. A reagent kit for diagnosing infection with Haemophilus inflenzae in a mammal comprising a LB1(F) peptide of claims 32-36.

62. A reagent kit for diagnosing infection with Haemophilus inflenzae in a mammal comprising an antibody of claim 54.
